SEQUENCE LISTING (14 sequences)

SEQ ID NO: 1 - protein, 13 aa, Virus
YNLRRGTALAIPQ

SEQ ID NO: 2 - protein, 13 aa, Virus
EGLRALLARSHVE

SEQ ID NO: 3 - protein, 15 aa, Virus
GVFVYGGSKTSLYNL

SEQ ID NO: 4 - protein, 13 aa, Virus
GPLRESIVCYFMV

SEQ ID NO: 5 - protein, 13 aa, Virus
ESIVCYFMVFLQT

SEQ ID NO: 6 - protein, 13 aa, Virus
YFMVFLQTHIFAE

SEQ ID NO: 7 - protein, 13 aa, Virus
AEVLKDAIKDLVM

SEQ ID NO: 8 - protein, 13 aa, Virus
KDLVMTKPAPTCN

SEQ ID NO: 9 - protein, 13 aa, Virus
NIRVTVCSFDDGV

SEQ ID NO: 10 - protein, 13 aa, Virus
PPWFPPMVEGAAA

SEQ ID NO: 11 - protein, 641 aa, Virus
MSDEGPGTGPGNGLGEKGDTSGPEGSGGSGPQRRGGDNHGRGRGRGRGRGGGRPGAPGGSGSGPRHRDGVRRPQKRPSCIGCKGTHGGTGAGAGAGGAGAGGAGAGGGAGAGGGAGGAGGAGGAGAGGGAGAGGGAGGAGGAGAGGGAGAGGGAGGAGAGGGAGGAGGAGAGGGAGAGGGAGGAGAGGGAGGAGGAGAGGGAGAGGAGGAGGAGAGGAGAGGGAGGAGGAGAGGAGAGGAGAGGAGAGGAGGAGAGGAGGAGAGGAGGAGAGGGAGGAGAGGGAGGAGAGGAGGAGAGGAGGAGAGGAGGAGAGGGAGAGGAGAGGGGRGRGGSGGRGRGGSGGRGRGGSGGRRGRGRERARGGSRERARGRGRGRGEKRPRSPSSQSSSSGSPPRRPPPGRRPFFHPVGEADYFEYHQEGGPDGEPDVPPGAIEQGPADDPGEGPSTGPRGQGDGGRRKKGGWFGKHRGQGGSNPKFENIAEGLRALLARSHVERTTDEGTWVAGVFVYGGSKTSLYNLRRGTALAIPQCRLTPLSRLPFGMAPGPGPQPGPLRESIVCYFMVFLQTHIFAEVLKDAIKDLVMTKPAPTCNIRVTVCSFDDGVDLPPWFPPMVEGAAAEGDDGDDGDEGGDGDEGEEGQE

SEQ ID NO: 12 - protein, 9 aa, Virus
YNLRRGTAL

SEQ ID NO: 13 - protein, 10 aa, Virus
YNLRRGTALA

SEQ ID NO: 14 - protein, 10 aa, Virus
NLRRGTALAI

(19) United States
(12) Patent Application Publication
(10) Pub. No.: US 2004/0141995 A1
(43) Pub. Date: Jul. 22, 2004

(54) MHC class I-restricted and MHC class II-restricted EBNA1 peptides

(76) Inventors: Rongfu Wang, Houston, TX (US); Kuishin Voo, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 10/732,694

(22) Filed: Dec. 10, 2003

Related U.S. Application Data
(60) Provisional application No. 60/432,319, filed on Dec. 10, 2002.

(51) Int. Cl. C12Q 1/68; C07H 21/04; A61K 39/12; C12N 7/00; C12N 5/08

(52) U.S. Cl. 424/186.1; 435/6; 435/69.1; 435/235.1; 435/372; 530/350; 536/23.2

(57) ABSTRACT

The present invention is directed to HLA-DP3 and HLA-B8 restricted peptides from EBNA1. Furthermore, the present invention relates to generating T cells specific for EBNA1 or antigen-presenting cell that present EBNA1 peptides. The present invention is also directed to methods for stimulating effector cell responses for cellular immunotherapy. Cellular immunotherapy can successfully prevent or treat various viral infections and tumors related to Epstein-Barr virus, such as Hodgkin's lymphoma. The invention is also directed to vaccines including EBNA1 peptide epitopes.

### CROSS-REFERENCE TO RELATED APPLICATIONS

This application claims priority to U.S. Provisional Application No. 60/432,319, which was filed Dec. 10, 2002, and which is hereby incorporated by reference in its entirety.

### STATEMENT REGARDING FEDERALLY SPONSORED RESEARCH OR DEVELOPMENT

The present invention was supported in part by the grant NIH P01 CA94237. Therefore, the United States Government may have certain rights in the invention.

### TECHNICAL FIELD

The field of this invention relates to immunology, molecular biology, and virology. Specifically, the invention describes immunological peptides of an Epstein-Barr virus specific protein, EBNA1. Further, the invention describes vaccines and treatments for Epstein-Barr virus and related diseases.

### BACKGROUND OF THE INVENTION

Epstein-Barr virus (EBV), a human gamma herpesvirus with tropism for B cells, has been implicated in the pathogenesis of a variety of human tumors, including Burkitt's lymphoma (BL), post transplant lymphoproliferative disorder (PTID), nasopharyngeal carcinoma (NPC) and Hodgkin's disease (HD) (Kieffet al. 1995, Rickinson et al. 1997).

Current combined modality therapy for Hodgkin Disease (HD), a pediatric lymphoma, results in a 60-95% cure rate, but relapsed HD carries a poor prognosis and the therapy is very toxic, resulting in second malignancies at a young age in 20% of survivors. At least 50% of HD patients exhibit EB virus in their malignant cells.

Burkitt's lymphoma is the most common childhood tumor in equatorial Africa, but is very rare in children in Western countries. Burkitt's lymphoma has been diagnosed in approximately 2% of AIDS patients.

Among the genes responsible for the growth-transforming function of EBV, EBNA1 is the only viral gene that is detected in all EBV-associated tumors including BL, NPC, PTID and HD (Rowe et al. 1987, Khanna et al. 1999). Other viral antigens such as the immunodominant EBNAs 3a, 3b and 3c are expressed only in type 3 tumors such as PTID, while two other antigens, latent membrane proteins LMP1 and LMP2, are expressed in type 2 tumors such as NPC and HD, but not in BL tumor (type I tumor). Thus, it appears that EBNA1 is a potentially important immune target for cancer immunotherapy.

Because of its important role in many EBV-associated cancers, EBNA1 has been suggested as an important target for immunotherapy. However, efforts by several groups to identify MHC class I-restricted peptides from EBNA1 have proved disappointing. For example, EBNA1-specific CD8⁺ T cells could be generated from human peripheral blood mononuclear cells (PBMCs) after in vitro stimulation with EBV-transformed B-lymphoblastoid cell lines (LCLs) pulsed with the exogenous EBNA1 protein, but they failed to recognize virally infected target cells such as LCLs or BL tumor cells. Previous failures to generate CD8⁺ T cells capable of recognizing naturally processed EBNA1 peptide on EBV-positive cells has been attributed to the inhibitory effect of the Gly-Ala repeat domain within EBNA1 on MHC class I-restricted antigen processing and presentation.

Studies from animal models and human clinical trials have demonstrated that CD4⁺ T cells play a central role in orchestrating host immune responses against cancer and infectious diseases (Kalams et al. 1998, Zajac et al. 1998, Wang et al. 2001). Indeed, CD4⁺ T cells consistently respond to the EBNA1 antigen in healthy donors and are capable of recognizing EBV-transformed B-lymphoblastoid cell lines (LCLs) (Munz et al. 2000, Nikiforow et al. 2001). To evaluate immune responses against EBNA1, EBNA3C, LMP1 and LMP2, several CD4⁺ T cell lines were generated from human peripheral blood mononuclear cells (PBMCs) after in vitro stimulation with dendritic cells pulsed with the corresponding purified proteins (Leen et al. 2001). Among the viral antigens tested, EBNA1 elicited the strongest CD4⁺ T-cell response, but these peptide-specific CD4⁺ T cells were not capable of recognizing naturally processed EBNA1 peptides on LCLs (Leen et al. 2001).

Methods for using isolated MHC complexes in the detection, quantification and purification of T-cells which recognize particular antigens have been studied for use in diagnostic and therapeutic applications. By way of example, early detection of T-cells specific for a particular autoantigen would facilitate the early selection of appropriate treatment regimes. The ability to purify antigen-specific T-cells would also be of great value in adoptive immunotherapy. Adoptive immunotherapy involves the removal of T-cells from a cancer patient, expansion of the T-cells in vitro and then reintroduction of the cells to the patient. Isolation and expansion of cancer specific T-cells with inflammatory properties would increase the specificity and effectiveness of such an approach.

The genes encoding the various proteins that constitute the MHC complexes have been extensively studied in humans and other mammals. In humans, MHC molecules (with the exception of class I β2-microglobulin) are encoded in the HLA region, which is located on chromosome 6 and constitutes over 100 genes. There are 3 class I MHC α protein loci, termed HLA-A, -B and -C. There are also 3 pairs of class II MHC α and β chain loci, termed HLA-DR(A and B), HLA-DP(A and B), and HLA-DQ(A and B).

Human CD4⁺ T cells consistently and predominantly respond to EBNA1 (Munz et al. 2000, Leen et al. 2001), illustrating that this antigen may be important target for immunotherapy. Because each of the dominant HLA-DR alleles accounts for only 10-20% of the general population, it can be deduced that T-cell peptides presented by different MHC class II molecules, including DP and DQ molecules, are present within the EBNA1 protein. To date, only three EBNA1 peptides presented by HLA-DR1, DR11 and DR15 have been identified, but these peptide-specific CD4⁺ T cells failed to recognize naturally processed epitopes on autologous LCLs or BL tumor cells (Leen et al. 2001, Khanna et al. 1995).

Adoptive therapy of EBV positive HD patients with EBV-specific CTLs has shown evidence of immune function and antitumor activity, but the overall immune responses were not sufficient to eradicate tumor cells (Roskrow et al. 1998, Heslop et al. 1999). Effective immunotherapy against EBV-associated malignancies should be aided by identifying MHC class II-restricted peptides from EBNA1 or other EBV-tumor associated antigens for use in cancer vaccines.

Although CD4⁺ T cells have often been generated from human PBMCs against putative tumor antigens or peptides, in many cases tumor reactivity could not be demonstrated due to either the low affinity of the T cells or the failure of tumor cells to present naturally processed peptides on their surface (Wang et al. 1999). Indeed, EBNA1 peptide-specific CD4⁺ T cells have been generated from human PBMCs after in vitro stimulation, but have failed to recognize autologous LCLs. T cell reactivity was found only when autologous LCL cells were preloaded with EBNA1 protein or pulsed with EBNA1 peptides (Leen et al. 2001, Khanna et al. 1995).

In a previous study it was shown that Ii-targeting can significantly enhance MHC class II antigen processing and presentation (Wang et al. 1999), and may override the requirement for other components in antigen presentation. It should be noted, however, that while tumor antigen MAGE-3 was recently identified as a MHC class II epitope presented by HLA-DR13 molecules, CD4⁺ T cells recognized only DR13⁺ B cells transfected with Ii-fused MAGE cDNA, not DR13⁺ B cells transfected with the full-length MAGE-3 cDNA (Chaux et al. 1999).

It was previously reported a CD4⁺ T cell line that can recognize NY-ESO-1 peptides in the context of HLA-DP4 molecules, even though this peptide contains an HLA-DR4 binding motif based on computer predictions and T cell reactivity experiments using HLA-DR4-transgenic mice (Zeng et al. 2001, Khanna et al. 1995). Hence, HLA-DP molecules may possess some features of the HLA-DR4-peptide binding motif. Alternatively, this could be due to the intrinsically promiscuous binding properties of MHC class II-restricted peptides (Zarour et al. 2002).

Munz et al. reported that CD4⁺ T cells generated in vitro could recognize EBV-positive LCL cells (Munz et al. 2000). These CD4⁺ T cells were later shown to recognize an HLA-DR1-restricted EBNA1 P₅₁₄₋₅₂₇ peptide (Paludan, et al. 2002), although this peptide is identical to the one previously described by Khanna and colleagues (Khanna et al. 1995).

### BRIEF SUMMARY OF THE INVENTION

An embodiment of the invention is an EBNA1 epitope comprising SEQ ID NO:1 or SEQ ID NO:12. In specific embodiments, the epitope is presented by MHC class I or MHC class II molecules.

In further specific embodiments, the epitope presented by MHC class II molecules is HLA-DP3-restricted and the epitope presented by MHC class I molecules is HLA-B8-restricted. In a specific embodiment of the invention, the epitope is presented by an Epstein-Barr infected B lymphocyte. In one embodiment, the epitope stimulates both CD4+ and CD8+ T cells.

An embodiment of the invention is a T cell comprising a CD4+ T cell or a CD8+T cell.

An embodiment of the invention is a method for stimulating T cells specific for an EBNA1 epitope comprising the step of contacting said T cells under conditions and for a time sufficient to permit the stimulation of said T cells, with at least one component selected from the group consisting of: the EBNA1 epitope, an antigen-presenting cell that recombinantly expresses and presents the EBNA1 epitope, and an antigen-presenting cell that expresses the endogenously processed EBNA1 epitope.

In specific embodiments, the antigen-presenting cell comprises a B lymphocyte or dendritic cell.

An embodiment of the invention is an isolated T cell population. Another embodiment of the invention is an immunological composition comprising T cells.

An embodiment of the invention is a method for stimulating an immune response in a patient, comprising the step of administering to the patient an immunological composition comprising a T cell population.

An embodiment of the invention is a method for expanding T cells specific for an EBNA1 epitope.

An embodiment of the invention is a method of treating human lymphoproliferative disorders. In specific embodiments, the lymphoproliferative disorder is Burkitt's lymphoma or Hodgkin's lymphoma.

An embodiment of the invention is a fusion protein comprising SEQ ID NO:1 or SEQ ID NO:12 and a domain that enhances MHC class II processing. The domain that enhances MHC class II processing comprises the invariant chain protein in specific embodiments.

An embodiment if the invention is a recombinant expression vector comprising an isolated nucleic acid sequence encoding SEQ ID NO:1 or SEQ ID NO:12 and at least one gene encoding a co-immunostimulatory molecule.

An embodiment if the invention is a method of treating a person infected with Epstein-Barr virus. In specific embodiments, the method further comprises the co-administration of at least one antigen-presenting cell.

An embodiment of the invention is a method for stimulating an immune response in a patient comprising the step of administering to said patient an immunological composition comprising at least one antigen presenting cell that presents an EBNA1 epitope comprising SEQ ID NO:1 or SEQ ID NO:12. In specific embodiments, the antigen presenting cell is a dendritic cell and/or the antigen presenting cell stimulates CD4+ and CD8+ T cells.

An embodiment of the invention is a nucleic acid vaccine comprising an EBNA1 epitope. In another embodiment, the invention comprises an isolated nucleic acid sequence comprising an EBNA1 epitope.

### BRIEF DESCRIPTION OF THE DRAWINGS

For a more complete understanding of the present invention, reference is now made to the following descriptions taken in conjunction with the accompanying drawings wherein:

FIG. 1A and FIG. 1B describe identification of T cell epitopes within EBNA1. FIG. 1A shows the generation of T cells from PBMCs from donor P after in vitro stimulation with synthetic peptides from EBNA1. FIG. 1B describes T cell recognition of the P3-W4 T cell line from donor P against 1359mel cells pulsed with EBNA1. P₅₁₈₋₅₃₀ (SEQ ID NO:1) peptide was specifically blocked by anti-HLA-class II and anti-HLA-DP antibodies;

FIG. 2A and FIG. 2B show the characterization of EBNA1-specific T cells. FIG. 2A, shows the recognition of GA876 BL tumor cells by CD4⁺T cell clones derived from P3-W4 T cell line. FIG. 2B illustrates FACS analysis of P3-B7 T cell clone for CD4 expression;

FIG. 3A and FIG. 3B describe the recognition of LCLs and EBV positive BL tumor cells by P3-B7 CD4⁺ T cells. FIG. 3A shows P3-B7 CD4⁺ T cell recognition of LCLs. T cell recognition was performed by co-culturing at an effector:target ratio of 1:1. IFN-γ secretion was measured by an ELISA kit. LCL7 and AG876 BL are DP3 positive based on DNA sequence analysis. HLA-DP typing of other cell lines is unknown. LCL1359 cells are HLA-DP3 negative. FIG. 3B shows T cell recognition of AG876 BL cells was inhibited by antibodies against MHC class II and HLA-DP molecules;

FIG. 4A, FIG. 4B, and FIG. 4C show the presentation of EBNA1 by HLA-DPA1*01031/B1*0301. FIG. 4A shows that P3-B7 CD4⁺ T cells recognize EBNA1 protein in the context of HLA-DP3 (DPA1*01031 and HLA-DPB1*0301) molecules. FIG. 4B is an evaluation of HLA-DP1, HLA-DP3 and HLA-DP4 molecules for their ability to present the EBNA1 peptide to T cells. Both autologous and AG876 tumor-derived HLA-DP3 cDNAs were capable of presenting the EBNA1 peptide to T cells. FIG. 4C shows full-length EBNA1 is presented to P3-B7 CD4⁺ T cells;

FIG. 5A and FIG. 5B show generation of EBNA1-specific T cells. FIG. 5A shows T cells that were generated from HLA-B8 expressing PBMCs of donor M after in vitro stimulation with synthetic peptides from EBNA1. Peptides other than those used for repeated stimulations served as negative controls. For T cell recognition assays, peptides were pulsed onto 1359mel target cells and co-cultured with T cells overnight. FIG. 5B illustrates that T cell recognition by T cell line M3-W1 from donor M of 1359mel cells pulsed with EBNA1 P₅₁₈₋₅₃₀ (SEQ ID NO:1) peptide was specifically inhibited by antibody against MHC class I molecules. T cell recognition assays were performed at an E/T ratio of 1:1. All results are expressed as IFN-γ release in pg/ml and are the averages of duplicate values. All antibodies were used at a final concentration of 20 μg/ml each;

FIG. 6A, FIG. 6B, FIG. 6C, and FIG. 6D show characterization of EBNA1-specific T cells. FIG. 6A depicts recognition of LCL 111 by T cell clones derived from the M3-W1 T-cell line. FIG. 6B illustrates FACS analysis of M3-W1-B9 T cells for CD8 expression. T cells were stained with anti-CD4-PE or anti-CD8-FITC. Positive staining for CD8 T cells is shown as an open histogram and control antibody staining is represented as a shaded histogram. FIG. 6C shows identification of minimal EBNA1 T cell epitope for MHC class I binding. Four different peptides were made and pulsed onto 1359mel cells at 10 μM concentration. After washing, the peptide-pulsed cells were co-cultured with T cells overnight. IFN-γ release was determined from culture supernatants. FIG. 6D shows an =EBNA1 P₅₁₈₋₅₂₆ (SEQ ID NO:12) peptide titration experiment for M3W1-B9 T cell recognition. EBNA1 P₅₁₈₋₅₂₆ (SEQ ID NO:12) peptide at various concentrations were pulsed on 1359mel cells and used as target cells to stimulate T cells. A control peptide EBNA1 P₅₇₂₋₅₈₄ (SEQ ID NO:7) was also used at various concentrations;

FIG. 7A, FIG. 7B, and FIG. 7C show the natural processing and presentation of EBNA1 to M3-W1-B9 CD8⁺ T cells. FIG. 7A illustrates recognition of full-length EBNA1 transfected 1359mel cells by M3-W1-B9 T cells. 1359mel cells were transfected with 200 ng of EBNA1-GFP or EBNA1-GAr-del-GFP plasmid DNAs using LipofectAMINE. FIG. 7B shows recognition of 1359 fibroblasts transfected with EBNA1-GFP by M3-W1-B9 T cells. 1359 fibroblasts were transfected with EBNA1 plasmid DNA by electroporation. T cell assays were performed at an effector:target ratio of 2:1. FIG. 7C shows T cells recognition of autologous PBMCs infected with retroviral/EBNA1-GFP;

FIG. 8A, FIG. 8B, FIG. 8C, and FIG. 8D show that HLA-B8 molecule functions as a restriction element for M3-W1-B9 CD8⁺ T cells. FIG. 8A shows T cell recognition of peptide-pulsed HLA-B8-expressing cell lines. FIG. 8B shows identification of HLA-B8 molecule as a restriction element for T cell recognition. HEK293 cells co-transfected with HLA-B8 plus full-length EBNA1-GFP or EBNA1-GAr-del-GFP cDNAs (with GAr domain deleted) were tested for recognition by M3-W1-B9 CD8⁺ T cells. Positive and negative signs indicate co-transfection of target cells in the presence or absence of HLA-B8 cDNA, respectively. FIG. 8C shows natural processing and presentation of the native form of EBNA1 for T cell recognition. HEK293 cells co-transfected with full-length EBNA1 and HLA-B8 cDNAs were co-cultured with M3-W1-B9 CD8⁺ T cells over night. IFN-γ secretion from T cells was determined by ELISA. FIG. 8D shows endogenous generation of HLA-B8-restricted EBNA1 peptide for T cell recognition. HEK293 cells transfected with HLA-B8 cDNA were mixed with HEK293 cells transfected with EBNA1-GFP cDNA at a 1:1 ratio. The mixed cells were then co-cultured with M3-W1-B9 CD8⁺ T cells overnight. IFN-γ release from CD8⁺ T cell was measured from culture supernatants;

FIG. 9A, FIG. 9B, and FIG. 9C illustrate specific lysis of HLA-B8-matched EBV-transformed LCLs by M3-W1-B9 CD8⁺ T cells. FIG. 9A shows recognition of HLA-B8 matched LCLs by M3-W1-B9 CD8⁺ T cells. LCLs were co-cultured with M3-W1-B9 CD8⁺ T cells at an E/T ratio of 1:1. Mismatched LCLs were used as negative controls. FIG. 9B shows specific lysis of HLA-B8-matched LCL 111 by CD8⁺ T cells at different E/T ratios. LCL 1 was used as a negative control. LCL cells were labeled with ⁵¹chromium. Cytolysis by CD8⁺ T cells was determined in a 16-h chromium release assay. FIG. 9C shows cold-target inhibition of recognition of LCL 111 cells by M3-W1-B9 CD8⁺ T cells. Lysis of LCLs by M3-W1-B9 CD8⁺ T cells was specifically inhibited when EBNA1 P₅₁₈₋₅₂₆-pulsed cold LCL 111 targets were used. Lysis was tested with an effector-to-hot target ratio of 40:1. Cold LCL 111 target cells were pulsed with EBNA1 P₅₁₈₋₅₂₆ or EBNA1-P₅₇₂₋₅₈₄ peptide at 1 μM concentration and were mixed with hot targets at a ratio of 4:1;

FIG. 10A and FIG. 10B show recognition of the EBNA1-P₅₁₈₋₅₃₀ (SEQ ID NO:1)peptide by CD4⁺ and CD8⁺ T cells. FIG. 10A depicts the alignment of HLA-DR1-, -DP3-and -B8-restricted peptides. FIG. 10B shows recognition of peptide-pulsed target cells by three different HLA-B8, -DR1 and -DP3-restricted T cell lines/clones. The HLA-DR1-restricted A4.E116 CD4⁺ T cells recognized an HLA-DR1-restricted EBNA1 peptide;

FIG. 11A, FIG. 11B, and FIG. 11C illustrate generation of EBNAL-P₅₁₈₋₅₂₆ (SEQ ID NO:12) peptide-specific T cells from HLA-B8 expressing PBMCs. FIG. 11A shows the detection of EBNA1-P₅₁₈₋₅₂₆ (SEQ ID NO:12) peptide reactive T cells from HLA-B8-positive donor PBMCs. 1×10⁵ PBMCs were seeded per well and experiments were performed in quadruplicate wells. An HLA-A2-restricted NY-ESO-1 peptide severed as a control. HLA-mismatched donor 5 and an HLA-B8 positive donor PBMCs 4 that is seronegative for EBV were also included. FIG. 11B shows recognition of LCL 1088 by CD8⁺ T cell clones from the PBMCs of donor 3. T cells generated from PBMCs were stimulated with EBNA1-P₅₁₈₋₅₂₆ (SEQ ID NO:12) peptide as described in FIG. 1. Six CD8⁺ T cell clones were generated from two T cell lines and were capable of recognizing HLA-B8 expressing LCL 1088 target cells. FIG. 11C shows D1-B11 CD8⁺ T cell recognition of HLA-B8-matched LCLs. LCLs were co-cultured with T cells at an E/T ratio of 1:1;

FIG. 12A, FIG. 12B, and FIG. 12C show specific inhibition of T cell recognition of EBNA1 by proteasomes inhibitors. FIG. 12A shows blocking of T cell recognition of EBNA1 by a ZAL proteasome inhibitor. HEK 293 cells co-transfected with EBNA1-GFP were treated with various concentrations of ZAL inhibitor for 10 h. After washing, cells were incubated with T cells overnight for IFN-γ release assays. Various dilutions of DMSO were used as controls. T cell activity in the absence of inhibitor was used as 100% activity. Two CD4⁺ T cells were used to demonstrate the specificity of ZAL inhibitor. TIL102 and P3-B7 CD4⁺T cells able to recognize 102mel and HEK293/DP3/Ii-EBNA1 target cells, respectively, were not inhibited by ZAL. FIG. 12B shows inhibition of M3-W1-B9 CD8⁺ T cell recognition of 1359mel target cells stably expressing EBNA1-GFP by lactacystin proteasome inhibitor. The lactacystin inhibitor did not affect recognition of 102mel tumor cells by TIL102 CD4⁺ cells. FIG. 12C shows blocking of MHC class II antigen processing by chloroquine. Inhibition of T cell recognition of 102mel cells by TIL102 CD4⁺ was observed after treatment with chloroquine in a dose-dependent fashion. By contrast, T cell recognition of LCL111 and HEK293 transfected with HLA-B8 and EBNA1-GFP cDNAs was not significantly affected after the treatment of chloroquine;

FIG. 13A and FIG. 13B show inhibition of T cell recognition of EBNA1 by protein synthesis inhibitors. FIG. 13A shows specific inhibition of M3-W1-B9 CD8⁺ T cell recognition of HEK293/B8/EBNA1-GFP target cells by an irreversible protein synthesis inhibitor emetine. HLA-B8 expressing HEK293/EBNA1-GFP target cells were treated with an emetine inhibitor at three concentrations for 1h. After three washings, cells were co-cultured with M3-W1-B9 CD8⁺ T cells overnight for IFN-γ release assays. Similar experiments were performed for the treatment of cells with cycloheximide or puromycin. HLA-B8 positive 1359 cells with the EBNA1-P₅₁₈₋₅₂₆ peptide after the treatment of 1359mel cells were pulsed with with 3 different concentration of emetine. FIG. 13B illustrates the determination of the sensitivity of recognition of TRP2-specific CD8 T cells to the treatment with emetine. 1363mel cells were treated with 3 different concentrations of emetine. After 3 washes, the cells were co-cultured with TRP2-specific CD8 ⁺ T cells. The treated cells pulsed with a TRP2 peptide were used to examine the effect of emetine on recognition of MHC class I/TRP2 complexes on the cell surface; and

FIG. 14A, FIG. 14B, and FIG. 14C show the requirement of serine proteases for the generation of EBNA1 T cell epitope. FIG. 14A shows specific inhibition of M3-W1-B9 CD8⁺ T cell recognition of HEK293/B8/EBNA1-GFP target cells by protease inhibitors. Target cells were incubated with various protease inhibitors for 2 h, washed and co-cultured with CD8⁺ T cells overnight for IFN-γ release assays. Solvents used to solubilize inhibitors were also used as controls. CD8⁺ T cell recognition of target cells was inhibited by treatment with TPCK and AEBSF inhibitors. FIG. 14B shows the effect of protease inhibitors on recognition of 1359mel cells by 1359mel-specific CD8⁺ T cells. FIG. 14C shows dose-dependent inhibition of M3-W1-B9 CD8⁺ T cell recognition of HEK293/B8/EBNA1-GFP target cells by protease inhibitors. Target cells were treated with different concentrations of protease inhibitors. After washes, the treated cells were co-cultured with M3-W1-B9 CD8⁺ T cells overnight for IFN-γ release assays.

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms.

As used herein, the expressions “cell”, “cell line” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

As used herein, “co-administration” may refer to joint administration to a patient such that two therapeutic, pharmacologic, immunologic, or other useful compositions may be administered at the same time. The compositions may be administered in the same injection, or one right after the other. The order of administration is not limited to a particular order. Additionally, “co-administration” also refers to joint administration to a patient during a single therapeutic regimen, which may take place over the course of hours, days, weeks, or months.

As used herein, an “epitope” is defined as a chemical structure capable of eliciting an immune cell response and of being specifically recognized by molecules of immune recognition (eg., immunoglobulins, T-cell receptors, etc.). Specifically, an “epitope” or an “EBNA1 epitope” refers an epitope comprising SEQ ID NO:1 or SEQ ID NO:12.

As used herein, “Human Leukocyte Antigen” or “HLA” is a human class I or class II Major Histocompatibility Complex (MHC) protein (see, e.g., Stites, et al., Immunology, 8.sup.TH Ed., Lange Publishing, Los Altos, Calif. (1994).

As used herein, presentation by MHC molecules occurs when a peptide fragment from a protein antigen taken by the antigen presenting cell (APC) becomes bound to proteins encoded in the major histocompatibility gene complex. The MHC-peptide complex is first formed in intracellular vesicles and is then transported to the plasma membrane, where it is recognized by T cells. Both the two major families of the MHC proteins, the class I and II, are peptide-binding proteins. The class II-MHC molecules bind primarily to peptides derived from the vacuolar digestion of internalized proteins. The MHC molecules constitute a protein system that rescues peptides from extensive intracellular degradation. The APC uses MHC-molecules to present antigenic determinants to the T cell system. An example of a protein involved in MHC protein presentation is the invariant chain protein.

The present invention includes a method of enhancing the immune response in a subject comprising the steps of contacting one or more lymphocytes with an HLA-DP3 or HLA-B8-restricted EBNA1 peptide antigenic composition, wherein the antigen comprises as part of its sequence a sequence comprising YNLRRGTALAIPQ (SEQ ID NO: 1), YNLRRGTAL (SEQ ID NO: 12), or an immunologically functional equivalent thereof. As used herein, an “antigenic composition” may comprise an antigen (e.g., a peptide or polypeptide), a nucleic acid encoding an antigen (e.g., an antigen expression vector), or a cell expressing or presenting an antigen. SEQ ID NO:11 is an example of a full-length EBNA1 peptide. One skilled in the art realizes that homologous variants of this protein are also appropriate sources for antigenic or immunogenic determinants. Furthermore, one skilled in the art knows that there are multiple strains of EBV, any of which may be sources of EBNA1. Examples of EBV strains are ag876, p3hr-1, CAO, and B95-8.

In other embodiments, the antigenic composition is in a mixture that comprises an additional immunostimulatory agent, or co-immunostimulatory agent, or nucleic acids encoding such an agent. Immunostimulatory agents include but are not limited to an additional antigen, an immunomodulator, an antigen presenting cell or an adjuvant. In other embodiments, one or more of the additional agent(s) is covalently bonded to the antigen or an agent, in any combination.

In certain embodiments the one or more lymphocytes are from an animal, such as a human. In certain embodiments, the animal is a human cancer patient and more preferably a human Hodgkins lymphoma patient or a human Burkitt's lymphoma cancer patient. In a preferred aspect, the one or more lymphocytes comprise a T-lymphocyte. In one facet, the T-lymphocyte is a cytotoxic T-lymphocyte.

The enhanced immune response may be an active or a passive immune response. Alternatively, the response may be part of an adoptive immunotherapy approach in which lymphocyte(s) are obtained with from an animal (e.g., a patient), then pulsed with composition comprising an antigenic composition. In this embodiment, the antigenic composition may comprise an additional immunostimulatory agent or a nucleic acid encoding such an agent. The lymphocyte(s) may be obtained from the blood of the subject, or alternatively from tumor tissue to obtain tumor infiltrating lymphocyte(s). In certain preferred embodiments, the lymphocyte(s) are peripheral blood lymphocyte(s). In a preferred embodiment, the lymphocyte(s) be administered to the same or different animal (e.g., same or different donors). In a preferred embodiment, the animal (e.g., a patient) has or is suspected of having a cancer, such as for example, Hodgkin's lymphoma. In other embodiments the method of enhancing the immune response is practiced in conjunction with a cancer therapy, such as for example, a cancer vaccine therapy.

Transformation of B cells by EBV is generally established by initializing a culture of peripheral blood lymphocytes, infecting them with EBV, and monitoring transformation by cell division.

As is well known in the art, a given composition may vary in its immunogenicity. It is often necessary therefore to boost the host immune system, as may be achieved by coupling a peptide and/or polypeptide immunogen to a carrier. Exemplary and/or preferred carriers are keyhole limpet hemocyanin (KLH) and/or bovine serum albumin (BSA). Other albumins such as ovalbumin, mouse serum albumin and/or rabbit serum albumin can also be used as carriers. Means for conjugating a polypeptide to a carrier protein are well known in the art and/or include glutaraldehyde, m-maleimidobenzoyl-N-hydroxysuccinimide ester, carbodiimide and/or bis-biazotized benzidine.

As is also well known in the art, the immunogenicity of a particular immunogen composition can be enhanced by the use of non-specific stimulators of the immune response, known as adjuvants. Suitable adjuvants include all acceptable immunostimulatory compounds, such as cytokines, toxins and/or synthetic compositions.

Adjuvants that may be used include IL-1, IL-2, IL-4, IL-7, IL-12, γ-interferon, GMCSP, BCG, aluminum hydroxide, MDP compounds, such as thur-MDP and/or nor-MDP, CGP (MTP-PE), lipid A, and/or monophosphoryl lipid A (MPL). RIBI, which contains three components extracted from bacteria, MPL, trehalose dimycolate (TDM) and/or cell wall skeleton (CWS) in a 2% squalene/Tween 80 emulsion is also contemplated. MHC antigens may even be used. Exemplary, often preferred adjuvants include complete Freund's adjuvant (a non-specific stimulator of the immune response containing killed Mycobacterium tuberculosis), algammulin incomplete Freund's adjuvants, Gerbu Adjuvant, nitrocellulose adsorbed protein, Montanide ISA, Hunter'TiterMax and/or aluminum hydroxide adjuvant.

In addition to adjuvants, it may be desirable to coadminister biologic response modifiers (BRM), which have been shown to upregulate T cell immunity and/or downregulate suppressor cell activity. Such BRMs include, but are not limited to, Cimetidine (CIM; 1200 mg/d) (Smith/Kline, PA); low-dose Cyclophosphamide (CYP; 300 mg/m²) (Johnson/ Mead, N.J.), cytokines such as γ-interferon, IL-2, and/or IL-12 and/or genes encoding proteins involved in immune helper functions, such as B-7.

1. Stimulation or Expansion of T Cells

In certain embodiments, T-lymphocytes are specifically activated by contact with an antigenic composition comprising SEQ ID NO:1 or SEQ ID NO: 12. In certain embodiments, T-lymphocytes are activated (stimulated or expanded) by contact with an antigen presenting cell that is in contact with an antigen of the invention.

T cells express a unique antigen binding receptor on their membrane (T-cell receptor), which can only recognize antigen in association with major histocompatibility complex (MHC) molecules on the surface of other cells. There are several populations of T cells, such as T helper cells and T cytotoxic cells. T helper cells and T cytotoxic cells are primarily distinguished by their display of the membrane bound glycoproteins CD4 and CD8, respectively. Activated T helper cells secret various lymphokines, that are crucial for the activation of B cells, T cytotoxic cells, macrophages and other cells of the immune system. In contrast, a T cytotoxic cell that recognizes an antigen-MHC complex is activated and proliferates and differentiates into an effector cell called a cytotoxic T lymphocyte (CTL). CTLs eliminate cells of the body displaying antigen, such as virus infected cells and tumor cells, by producing substances that result in cell lysis.

The present invention deals with efficacious peptide epitope compositions that induce a therapeutic or prophylactic immune response when administered via various art-accepted modalities. These peptides can also be used diagnostically, e.g., to evaluate the immune response to an antigen. A complex of an HLA molecule and a peptidic antigen acts as the ligand recognized by HLA-restricted T cells (Buus, S. et al., Cell 47:1071, 1986; Babbitt, B. P. et al., Nature 317:359, 1985; Townsend, A. and Bodmer, H., Annu. Rev. Immunol. 7:601, 1989; Germain, R. N., Annu. Rev. Immunol. 11:403, 1993).

Various strategies can be utilized to evaluate immunogenicity, including: 1) Evaluation of primary T cell cultures from normal individuals (see, e.g., Wentworth, P. A. et al., Mol. Immunol. 32:603 (1995); Celis, E. et al., Proc. Natl. Acad. Sci. USA 91:2105 (1994); Tsai, V. et al., J. Immunol. 158:1796 (1997); Kawashima, I. et al., Human Immunol. 59:1 (1998)). This procedure involves the stimulation of peripheral blood lymphocytes (PBL) from normal subjects with a test peptide in the presence of antigen presenting cells in vitro over a period of several weeks. T cells specific for the peptide become activated during this time and are detected using, e.g., a ⁵¹Cr-release assay involving peptide sensitized target cells, and/or target cells that generate antigen endogenously; 2) Immunization of HLA transgenic mice (see, e.g., Wentworth, P. A. et al., J. Immunol. 26:97 (1996); Wentworth, P. A. et al., Int. Immunol. 8:651 (1996); Alexander, J. et al., J. Immunol. 159:4753 (1997)); in this method, peptides in incomplete Freund's adjuvant are administered subcutaneously to HLA transgenic mice. Several weeks following immunization, splenocytes are removed and cultured in vitro in the presence of test peptide for approximately one week. Peptide-specific T cells are detected using, e.g., a ⁵¹Cr-release assay involving peptide sensitized target cells and target cells expressing endogenously generated antigen; and 3) Demonstration of recall T cell responses from individuals exposed to the disease, such as immune individuals who were effectively treated and recovered from disease, and/or from actively ill patients (see, e.g., Rehermann, B. et al., J. Exp. Med. 181:1047 (1995); Doolan, D. L. et al., Immunity 7:97 (1997); Bertoni, R. et al., J. Clin. Invest. 100:503 (1997); Threlkeld, S. C. et al., J. Immunol. 159:1648 (1997); Diepolder, H. M. et al., J. Virol. 71:6011 (1997)). In applying this strategy, recall responses are detected by culturing PBL from subjects in vitro for 1-2 weeks in the presence of a test peptide plus antigen presenting cells (APC) to allow activation of “memory” T cells, as compared to “naive” T cells. At the end of the culture period, T cell activity is detected using assays for T cell activity including ⁵¹Cr release involving peptide-sensitized targets, T cell proliferation, or lymphokine release.

Also, an in vitro dehydrogenase release assay has been developed that takes advantage of a new fluorescent amplification system. This approach is sensitive, rapid, reproducible and may be used advantageously for mixed lymphocyte reaction (MLR). It may easily be further automated for large scale cytotoxicity testing using cell membrane integrity, and is thus considered in the present invention. In another fluorometric assay developed for detecting cell-mediated cytotoxicity, the fluorophore used is the non-toxic molecule alamarBlue. The alamarBlue is fluorescently quenched (i.e., low quantum yield) until mitochondrial reduction occurs, which then results in a dramatic increase in the alamarBlue fluorescence intensity (i.e., increase in the quantum yield). This assay is reported to be extremely sensitive, specific and requires a significantly lower number of effector cells than the standard ⁵¹Cr release assay.

In certain aspects, T helper cell responses can be measured by in vitro or in vivo assay with peptides, polypeptides or proteins. In vitro assays include measurement of a specific cytokine release by enzyme, radioisotope, chromaphore or fluorescent assays. In vivo assays include delayed type hypersensitivity responses called skin tests, as would be known to one of ordinary skill in the art.

2. Antigen Presenting Cells

In general, the term “antigen-presenting cell” can be any cell that accomplishes the goal of the invention by aiding the enhancement of an immune response (i.e., from the T-cell or -B-cell arms of the immune system) against an antigen (e.g., a sequence comprising SEQ ID NO:1, SEQ ID NO: 12, or a immunologically functional equivalent) or antigenic composition of the present invention. Such cells can be defined by those of skill in the art, using methods disclosed herein and in the art. Examples of antigen-presenting cells are B cells, dendritic cells, Langerhans cells, macrophages and monocytes, thymic dendritic cells, thymic endothelial cells, or vascular endothelial cells. Other antigen-presenting cells are inducible, and thus may present antigens in association with an inflammation response. Examples of such cells are fibroblasts and glial cells.

As is understood by one of ordinary skill in the art, and used in certain embodiments, a cell that displays or presents an antigen normally or preferentially with a class II major histocompatability molecule or complex to an immune cell is an “antigen-presenting cell.” Such cells, as described above, may consitutively express class II MHC complexes, or may inducibly express them. In certain aspects, a cell (e.g., an APC cell) may be fused with another cell, such as a recombinant cell or a tumor cell that expresses the desired antigen. Methods for preparing a fusion of two or more cells is well known in the art,. In some cases, the immune cell to which an antigen presenting cell displays or presents an antigen to is a CD4⁺T_{H} cell. Additional molecules expressed on the APC or other immune cells may aid or improve the enhancement of an immune response. Secreted or soluble molecules, such as for example, cytokines and adjuvants, may also aid or enhance the immune response against an antigen. Such molecules are well known to one of skill in the art, and various examples are described herein.

3. Vaccines

For an antigenic composition to be useful as a vaccine, an antigenic composition must induce an immune response to the antigen in a cell, tissue or animal (e.g., a human). As used herein, an “antigenic composition” may comprise an antigen (e.g., a peptide or polypeptide), a nucleic acid encoding an antigen (e.g., an antigen expression vector), or a cell expressing or presenting an antigen. In particular embodiments the antigenic composition comprises or encodes all or part of the sequences shown in SEQ ID NO:1 and/or SEQ ID NO: 12, or immunologically functional equivalents thereof. In other embodiments, the antigenic composition is in a mixture that comprises an additional immunostimulatory agent or nucleic acids encoding such an agent. Immunostimulatory agents include but are not limited to an additional antigen, an immunomodulator, an antigen presenting cell or an adjuvant. In other embodiments, one or more of the additional agent(s) is covalently bonded to the antigen or an immunostimulatory agent, in any combination. In certain embodiments, the antigenic composition is conjugated to or comprises HLA anchor motif amino acids.

In certain embodiments, an antigenic composition or immunologically functional equivalent, may be used as an effective vaccine in inducing an anti-EBNA1 humoral and/or cell-mediated immune response in an animal. The present invention contemplates one or more antigenic compositions or vaccines for use in both active and passive immunization embodiments.

A vaccine of the present invention may vary in its composition of proteinaceous, nucleic acid and/or cellular components. In a non-limiting example, a nucleic acid encoding an antigen might also be formulated with a proteinaceous adjuvant. Of course, it will be understood that various compositions described herein may further comprise additional components. For example, one or more vaccine components may be comprised in a lipid or liposome. In another non-limiting example, a vaccine may comprise one or more adjuvants. A vaccine of the present invention, and its various components, may be prepared and/or administered by any method disclosed herein or as would be known to one of ordinary skill in the art, in light of the present disclosure.

4. Epitopic Core Sequences

In another aspect, the invention provides a peptide or polypeptide comprising an “epitope-bearing” portion of a polypeptide of the invention. The epitope of this polypeptide portion is an immunogenic or antigenic epitope of a polypeptide of the invention. An “immunogenic epitope” is defined as a part of a protein that elicits an antibody response when the whole protein is the immunogen. These immunogenic epitopes are believed to be confined to a few loci on the molecule. A region of a protein molecule to which an antibody can bind is defined as an “antigenic epitope.” An epitope may also be a region of a peptide which is recognized by T cells when presented by MHC class I or class II molecules.

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein. Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals. Peptides that are extremely hydrophobic and those of six or fewer residues generally are ineffective at inducing antibodies that bind to the mimicked protein; longer, soluble peptides, especially those containing proline residues, usually are effective. Sutcliffe et al., supra, at 661. For instance, 18 of 20 peptides designed according to these guidelines, containing 8-39 residues covering 75% of the sequence of the influenza virus hemagglutinin HA1 polypeptide chain, induced antibodies that reacted with the HA1 protein or intact virus; and 12/12 peptides from the MuLV polymerase and 18/18 from the rabies glycoprotein induced antibodies that precipitated the respective proteins.

U.S. Pat. No. 4,554,101, (Hopp) incorporated herein by reference, teaches the identification and/or preparation of epitopes from primary amino acid sequences on the basis of hydrophilicity. Through the methods disclosed in Hopp, one of skill in the art would be able to identify epitopes from within an amino acid sequence.

One skilled in the art recognizes that numerous scientific publications have also been devoted to the prediction of secondary structure, and/or to the identification of epitopes, from analyses of amino acid sequences. Any of these may be used, if desired, to supplement the teachings of Hopp in U.S. Pat. No. 4,554,101.

Moreover, computer programs are currently available to assist with predicting antigenic portions and/or epitopic core regions of proteins. Examples include those programs based upon the Jameson-Wolf analysis, the program PepPlot®, and/or other new programs for protein tertiary structure prediction. Another commercially available software program capable of carrying out such analyses is MacVector (EBI, New Haven, Conn.).

Antigenic epitope-bearing peptides and polypeptides of the invention are therefore useful to raise antibodies, including monoclonal antibodies, that bind specifically to a polypeptide of the invention. Thus, a high proportion of hybridomas obtained by fusion of spleen cells from donors immunized with an antigen epitope-bearing peptide generally secrete antibody reactive with the native protein.

Antigenic epitope-bearing peptides and polypeptides of the invention designed according to the above guidelines preferably contain a sequence of at least seven, more preferably at least nine and most preferably between about 15 to about 30 amino acids contained within the amino acid sequence of a polypeptide of the invention. However, peptides or polypeptides comprising a larger portion of an amino acid sequence of a polypeptide of the invention, containing about 30 to about 50 amino acids, or any length up to and including the entire amino acid sequence of a polypeptide of the invention, also are considered epitope-bearing peptides or polypeptides of the invention and also are useful for inducing antibodies that react with the mimicked protein. Preferably, the amino acid sequence of the epitope-bearing peptide is selected to provide substantial solubility in aqueous solvents (i.e., the sequence includes relatively hydrophilic residues and highly hydrophobic sequences are preferably avoided); and sequences containing proline residues are particularly preferred.

The epitope-bearing peptides and polypeptides of the invention may be produced by any conventional means for making peptides or polypeptides including recombinant means using nucleic acid molecules of the invention. For instance, a short epitope-bearing amino acid sequence may be fused to a larger polypeptide which acts as a carrier during recombinant production and purification, as well as during immunization to produce anti-peptide antibodies. Epitope-bearing peptides also may be synthesized using known methods of chemical synthesis. For instance, Houghten has described a simple method for synthesis of large numbers of peptides, such as 10-20 mg of 248 different 13 residue peptides representing single amino acid variants of a segment of the HAI polypeptide which were prepared and characterized (by ELISA-type binding studies) in less than four weeks. This “Simultaneous Multiple Peptide Synthesis (SMPS)” process is further described in U.S. Pat. No. 4,631,211 to Houghten et al. (1986). In this procedure the individual resins for the solid-phase synthesis of various peptides are contained in separate solvent-permeable packets, enabling the optimal use of the many identical repetitive steps involved in solid-phase methods. A completely manual procedure allows 500-1000 or more syntheses to be conducted simultaneously. Houghten et al., supra, at 5134.

Further still, U.S. Pat. No. 5,194,392 to Geysen (1990) describes a general method of detecting or determining the sequence of monomers (amino acids or other compounds) which is a topological equivalent of the epitope (i.e., a “mimotope”) which is complementary to a particular paratope (antigen binding site) of an antibody of interest. More generally, U.S. Pat. No. 4,433,092 to Geysen (1989) describes a method of detecting or determining a sequence of monomers which is a topographical equivalent of a ligand which is complementary to the ligand binding site of a particular receptor of interest. Similarly, U.S. Pat. No. 5,480,971 to Houghten, R. A. et al. (1996) on Peralkylated Oligopeptide Mixtures discloses linear C.sub.1-C.sub.7-alkyl peralkylated oligopeptides and sets and libraries of such peptides, as well as methods for using such oligopeptide sets and libraries for determining the sequence of a peralkylated oligopeptide that preferentially binds to an acceptor molecule of interest. Thus, non-peptide analogs of the epitope-bearing peptides of the invention also can be made routinely by these methods.

In further embodiments, major antigenic determinants of a polypeptide may be identified by an empirical approach in which portions of the gene encoding the polypeptide are expressed in a recombinant host, and/or the resulting proteins tested for their ability to elicit an immune response. For example, PCR™ can be used to prepare a range of peptides lacking successively longer fragments of the C-terminus of the protein. The immunoactivity of each of these peptides is determined to identify those fragments and/or domains of the polypeptide that are immunodominant. Further studies in which only a small number of amino acids are removed at each iteration then allows the location of the antigenic determinants of the polypeptide to be more precisely determined.

Another method for determining the major antigenic determinants of a polypeptide is the SPOTS™ system (Genosys Biotechnologies, Inc., The Woodlands, Tex.). In this method, overlapping peptides are synthesized on a cellulose membrane, which following synthesis and/or deprotection, is screened using a polyclonal and/or monoclonal antibody. The antigenic determinants of the peptides which are initially identified can be further localized by performing subsequent syntheses of smaller peptides with larger overlaps, and/or by eventually replacing individual amino acids at each position along the immunoreactive peptide.

5. Proteinaceous Antigens

It is understood that an antigenic composition of the present invention may be made by a method that is well known in the art, including but not limited to chemical synthesis by solid phase synthesis and purification away from the other products of the chemical reactions by HPLC, or production by the expression of a nucleic acid sequence (e.g., a DNA sequence) encoding a peptide or polypeptide comprising an antigen of the present invention in an in vitro translation system or in a living cell. Preferably the antigenic composition isolated and extensively dialyzed to remove one or more undesired small molecular weight molecules and/or lyophilized for more ready formulation into a desired vehicle. It is further understood that additional amino acids, mutations, chemical modification and such like, if any, that are made in a vaccine component will preferably not substantially interfere with the antibody recognition of the epitopic sequence.

A peptide or polypeptide corresponding to an antigenic determinant of EBNA1 of the present invention may be synthesized by methods known to those of ordinary skill in the art, such as, for example, peptide synthesis using automated peptide synthesis machines, such as those available from Applied Biosystems (Foster City, Calif.). Polypeptides are disclosed herein as amino acid residue sequences. Those sequences are written left to right in the direction from the amino to the carboxy terminus. In accordance with standard nomenclature, amino acid residue sequences are denominated by either a single letter or a three letter code as indicated below.

Longer peptides or polypeptides also may be prepared, e.g., by recombinant means. In certain embodiments, a nucleic acid encoding an antigenic composition and/or a component described herein may be used, for example, to produce an antigenic composition in vitro or in vivo for the various compositions and methods of the present invention. For example, in certain embodiments, a nucleic acid encoding an antigen is comprised in, for example, a vector in a recombinant cell. The nucleic acid may be expressed to produce a peptide or polypeptide comprising an antigenic sequence. The peptide or polypeptide may be secreted from the cell, or comprised as part of or within the cell.

6. Genetic Vaccine Antigens

In certain embodiments, an immune response may be promoted by transfecting or inoculating an animal with a nucleic acid encoding an antigen. One or more cells comprised within a target animal then expresses the sequences encoded by the nucleic acid after administration of the nucleic acid to the animal. Thus, the vaccine may comprise “genetic vaccine” useful for immunization protocols. A vaccine may also be in the form, for example, of a nucleic acid (e.g., a CDNA or an RNA) encoding all or part of the peptide or polypeptide sequence of an antigen. Expression in vivo by the nucleic acid may be, for example, by a plasmid type vector, a viral vector, or a viral/plasmid construct vector.

In preferred aspects, the nucleic acid comprises a coding region that encodes all or part of the sequences disclosed as SEQ ID NO:1 and/or SEQ ID NO: 12, or immunologically functional equivalents thereof. Of course, the nucleic acid may comprise and/or encode additional sequences, including but not limited to those comprising one or more immunomodulators or adjuvants. The nucleotide and protein, polypeptide and peptide encoding sequences for various genes have been previously disclosed, and may be found at computerized databases known to those of ordinary skill in the art. One such database is the National Center for Biotechnology Information's Genbank and GenPept databases.

7. Cellular Vaccine Antigens

In another embodiment, a cell expressing the antigen may comprise the vaccine. The cell may be isolated from a culture, tissue, organ or organism and administered to an animal as a cellular vaccine. Thus, the present invention contemplates a “cellular vaccine.” The cell may be transfected with a nucleic acid encoding an antigen to enhance its expression of the antigen. Of course, the cell may also express one or more additional vaccine components, such as immunomodulators or adjuvants. A vaccine may comprise all or part of the cell.

In particular embodiments, it is contemplated that nucleic acids encoding antigens of the present invention may be transfected into plants, particularly edible plants, and all or part of the plant material used to prepare a vaccine, such as for example, an oral vaccine. Such methods are described in U.S. Pat. Nos. 5,484,719, 5,612,487, 5,914,123, 5,977,438 and 6,034,298, each incorporated herein by reference.

8. Immunologically Functional Equivalents

As modifications and changes may be made in the structure of an antigenic composition of the present invention, and still obtain molecules having like or otherwise desirable characteristics, such immunologically functional equivalents are also encompassed within the present invention.

For example, certain amino acids may be substituted for other amino acids in a peptide, polypeptide or protein structure without appreciable loss of interactive binding capacity with structures such as, for example, antigen-binding regions of antibodies, binding sites on substrate molecules or receptors, DNA binding sites, or such like. Since it is the interactive capacity and nature of a peptide, polypeptide or protein that defines its biological (e.g., immunological) functional activity, certain amino acid sequence substitutions can be made in a amino acid sequence (or, of course, its underlying DNA coding sequence) and nevertheless obtain a peptide or polypeptide with like (agonistic) properties. It is thus contemplated by the inventors that various changes may be made in the sequence of an antigenic composition or underlying DNA, without appreciable loss of biological utility or activity.

As used herein, an “amino molecule” refers to any amino acid, amino acid derivative or amino acid mimic as would be known to one of ordinary skill in the art. In certain embodiments, the residues of the antigenic composition comprises amino molecules that are sequential, without any non-amino molecule interrupting the sequence of amino molecule residues. In other embodiments, the sequence may comprise one or more non-amino molecule moieties. In particular embodiments, the sequence of residues of the antigenic composition may be interrupted by one or more non-amino molecule moieties.

Accordingly, antigenic composition, particularly an immunologically functional equivalent of the sequences disclosed herein, may encompass an amino molecule sequence comprising at least one of the 20 common amino acids in naturally synthesized proteins, or at least one modified or unusual amino acid, including but not limited to those shown on [Table 1] below.

TABLE 1
Modified and Unusual Amino Acids

| Abbr. | Amino Acid |
|---|---|
| Aad | 2-Aminoadipic acid |
| Baad | 3-Aminoadipic acid |
| Bala | β-alanine, β-Amino-propionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| Baib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| Ahyl | Allo-Hydroxylysine |
| 3Hyp | 3-Hydroxyproline |
| 4Hyp | 4-Hydroxyproline |
| Ide | Isodesmosine |
| Aile | Allo-Isoleucine |
| MeGly | N-Methylglycine, sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

In term of immunologically functional equivalent, it is well understood by the skilled artisan that, inherent in the definition is the concept that there is a limit to the number of changes that may be made within a defined portion of the molecule and still result in a molecule with an acceptable level of equivalent immunological activity. An immunologically functional equivalent peptide or polypeptide are thus defined herein as those peptide(s) or polypeptide(s) in which certain, not most or all, of the amino acid(s) may be substituted.

In particular, where a shorter length peptide is concerned, it is contemplated that fewer amino acid substitutions should be made within the given peptide. A longer polypeptide may have an intermediate number of changes. The full length protein will have the most tolerance for a larger number of changes. Of course, a plurality of distinct polypeptides/peptides with different substitutions may easily be made and used in accordance with the invention.

9. Immunotherapeutic Agents

An immunotherapeutic agent generally relies on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (e.g., a chemotherapeutic, a radionuclide, a ricin A chain, a cholera toxin, a pertussis toxin, etc.) and serve merely as a targeting agent. Such antibody conjugates are called immunotoxins, and are well known in the art (see U.S. Pat. No. 5,686,072, U.S. Pat. No. 5,578,706, U.S. Pat. No. 4,792,447, U.S. Pat. No. 5,045,451, U.S. Pat. No. 4,664,911, and U.S. Pat. No. 5,767,072, each incorporated herein by reference). Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

10. Immune Stimulators

In a specific aspect of immunotherapy is to use an immune stimulating molecule as an agent, or more preferably in conjunction with another agent, such as for example, a cytokines such as for example IL-2, IL-4, IL-12, GM-CSF, tumor necrosis factor; interferons alpha, beta, and gamma; F42K and other cytokine analogs; a chemokine such as for example MIP-1, MIP-1beta, MCP-1, RANTES, IL-8; or a growth factor such as for example FLT3 ligand.

One particular cytokine contemplated for use in the present invention is tumor necrosis factor. Tumor necrosis factor (TNF; Cachectin) is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. Some infectious agents cause tumor regression through the stimulation of TNF production. TNF can be quite toxic when used alone in effective doses, so that the optimal regimens probably will use it in lower doses in combination with other drugs. Its immunosuppressive actions are potentiated by gamma-interferon, so that the combination potentially is dangerous. A hybrid of TNF and interferon-α also has been found to possess anti-cancer activity.

Another cytokine specifically contemplate is interferon alpha. Interferon alpha has been used in treatment of hairy cell leukemia, Kaposi's sarcoma, melanoma, carcinoid, renal cell cancer, ovary cancer, bladder cancer, non-Hodgkin's lymphomas, mycosis fingoides, multiple myeloma, and chronic granulocytic leukemia.

11. Passive Immunotherapy

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow. Preferably, human monoclonal antibodies are employed in passive immunotherapy, as they produce few or no side effects in the patient. However, their application is somewhat limited by their scarcity and have so far only been administered intralesionally. It may be favorable to administer more than one monoclonal antibody directed against two different antigens or even antibodies with multiple antigen specificity. Treatment protocols also may include administration of lymphokines or other immune enhancers.

12. Active Immunotherapy

In active immunotherapy, an antigenic peptide, polypeptide or protein, or an autologous or allogenic tumor cell composition or “vaccine” is administered, generally with a distinct bacterial adjuvant.

13. Adoptive Immunotherapy

In adoptive immunotherapy, the patient's circulating lymphocytes, or tumor infiltrated lymphocytes, are isolated in vitro, activated by lymphokines such as IL-2 or transduced with genes for tumor necrosis, and readministered. To achieve this, one would administer to an animal, or human patient, an immunologically effective amount of activated lymphocytes in combination with an adjuvant-incorporated antigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders were few compared to those who did not respond.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Cell Lines, Reagents and Antibodies

Burkitt's lymphoma cell lines AG876, Akata and Eli; EBV-transformed LCLs 1 to 7, 8, 111, 888, 1088, 1359, and EBV-DG75; melanoma cell lines 102mel, 586mel, 1359mel; 1359 fibroblasts; and HEK 293 cell lines were maintained in RPMI 1640 medium (Sigma-Aldrich) supplemented with 10% FCS growth medium (Gemini). Antibodies from Hybridoma HB55 (anti-DR), HB 95 (anti-class I), HB 144 (anti-DQ) and HB 145 (anti-class II) (American Type Culture Collection) were purified from culture supernatants of hybridoma. FITC-conjugated anti-CD4 and CD8⁺ were purchased from PharMingen by methods known in the art. EBNA1 protein was expressed in SF-9 cells and purified by methods known in the art. See for example Frappier et al. 1991.

Proteasome inhibitors Z-Ile-Glu(OtBu)-Ala-Leucinal (ZAL) and lactacystin were purchased from CALBIOCHEM. MHC class II pathway inhibitor chloroquine was purchased from Sigma-Aldrich. Protein synthesis inhibitors emetine, cycloheximide and puromycin were purchased from CALBIOCHEM. Protease inhibitors, including AEBSF, E-64, EST, Leupeptin, Pepstatin A, TLCK and TPCK were purchased from CALBIOCHEM. All chemicals were dissolved in solvents recommended by the manufacturers.

### Example 2

### HLA Typing of Donor Peripheral Blood Mononuclear Cells

The HLA serotypes and DNA genotypes of PBMCs from healthy human were determined by the National Institutes of Health HLA Laboratory. The HLA genotype of PBMC from donor M was HLA-A*01, 0201, B*08, DRβ1*0301, 0401, DQβ1*0201, 0301, DRβ3*0101, DRβ4*01; for donor P it was HLA-A*0201, 32, B*4001, 51, DRβ1*0401, 0801, DQβ1*0302, 04, DRβ04*0101; for donor Q it was HLA-A*01, 6802, B*15, 53, DRβ1*0401, 1302, DQβ1*0301, 0501, DRβ3*0301, DRβ4*0101; for donor S it was HLA-A*0301, 29, B*44, 4501, DRβ1*0401, 0701, DQβ1*0201, 0301, DRβ04*01; and for 1359mel cell line it was HLA-A*01, B*8, 40, CW*03, 07, DRβ1*0401, 17, DQβ1*02, 03, DRβ03*0101, β4*0101. The genotypes for donors #1, 2 and 4 was HLA-B*8, and for donor # 3 it was HLA-A*01, 30, B*8, 13, CW*06, 07, DRβ1*3, 7, DQβ1*02, DRβ3*01, 04*01.

The molecular typing of HLA-DP molecules for the PBMCs from donor P was performed by methods known to one with skill in the art. See for example Zeng et al. 2001. DNA sequences were searched against the IMGT-HLA database to determine the HLA-DP identity.

### Example 3

### Synthetic EBNA1 Peptides

Ten peptides encompassing B95.8 strain EBNA1 P₅₁₈₋₅₃₀ (YNLRRGTALAIPQ) (SEQ ID NO:1), P₄₈₃₋₄₉₅ (EGLRALLAR SHVE) (SEQ ID NO:2), P₅₀₆₋₅₂₀ (GVFVYGGSKTSLYNL) (SEQ ID NO:3), P₅₅₂₋₅₆₄ (GPLRESIVCYFMV) (SEQ ID NO:4), P₅₅₆₋₅₆₈ (ESIVCYFMVFLQT) (SEQ ID NO:5), P₅₆₁₋₅₇₃ (YFMVFLQTHIFAE) (SEQ ID NO:6), P₅₇₂₋₅₈₄ (AEVLKDAIKDLVM) (SEQ ID NO:7), P₅₈₀₋₅₉₂ (KDLVMTKPAPTCN) (SEQ ID NO:8), P₅₉₂₋₆₀₄ (NIRVTVCSFDDGV) (SEQ ID NO:9) and P₆₀₇₋₆₁₉ (PPWFPPMVEGAAA) (SEQ ID NO:10), were synthesized by standard fluorenyl-methoxycarbonyl chemistry and dissolved in dimethylsulfoxide (DMSO). The purity and molecular masses of peptides were determined by HPLC and mass spectrometry.

### Example 4

### Generation of Human CD4⁺ T Cell Lines and Clones

PBMCs from three donors (S, P, Q) were used for peptide stimulation in vitro in lymphocyte culture medium at 2×10⁵ cells per well in a flat-bottomed 96-well plate, as described (see Tan et al., 1999). Two weeks after stimulation, each subline was again screened for specific peptide reactivity. T cell reactivity was tested to determine the restriction element in the presence of anti-HLA-A, B and C, anti-HLA class II, HLA-DP, -DQ and -DR mAb at a 20 μg/ml of antibody concentration. T cell clones were generated from bulk T cell lines by the limiting dilution method, known to one with skill in the art. See for example Wang et al. 1998.

Human PBMCs from three HLA-DR4 expressing donors (M, Q, S) were used for peptide stimulation in vitro in lymphocyte culture medium at 1.5×10⁵ cells per well in a flat-bottom 96-well plate. See Voo et al., 2002 One T cell line from donor M was generated that showed specific T cell reactivity against peptide-pulsed 1359mel cells which was blocked by anti-MHC class I monoclonal antibody. T cell line M was further cloned using limiting dilution methods. See Wang et al., 1998.

### Example 5

### Transfection of EBNA1 Expression Constructs

Full length EBNA1, EBNA1-GFP and GAr-deleted EBNA1-GFP constructs as described in Tellam et al. 2001 were obtained from Judy Tellam and R. Khanna, University of Queensland, Brisbane, Australia. An expression vector pIi-EBNA1(aa₄₇₅₋₆₀₀) was constructed by subcloning full length EBNA1 into a pTSX expression vector to express as an Ii-fusion protein with a targeting sequence (amino acid 1-80) of invariant chain (Ii). HEK293 and 1359melcells were transfected with LipofectAMlNE reagent (Invitrogen, Carlsbad, Calif.). Transfection and T cell activity assay were performed by methods known in the art. See for example Zeng, et al. 2001, Wang and Wang et al. 1999.

### Example 6

### Cytokine Release, Cytotoxicity and ELISPOT Assays.

Human EBNA1-specific T cell clones were identified on the basis of their ability to release IFN-γ. Cytotoxicity and cold-target inhibition assays were performed as previously described (13). Briefly, LCL 111 hot target cells were labeled with 100 μCi Na₂⁵¹CrO4, either alone or in the presence of 1 μM of EBNA1 P₅₁₈₋₅₂₆ peptide for 8 h at 37° C. Target cells were washed three times with RPMI1640, counted, and then mixed with CD8⁺ T cells at the indicated E:T ratios. Chromium release was measured after 16 h incubation. In cold target inhibition assays, LCL 111 target cells were pulsed with 1 μM of EBNA1 P₅₁₈₋₅₂₆ or EBNA1-P₅₇₂₋₅₈₄ peptides for 90 min, washed three times with RPMI, counted and incubated with CD8⁺ T cells for 30 min before the addition of hot target cells. An effector to target ratio of 40:1 and a cold to hot target ratio of 4:1×10⁴ were used in these assays. The percentage of specific lysis was determined from the equation [(cpm experimental well-cpm spontaneous release)/(cpm maximum release-cpm spontaneous release)]×100%. The ELISPOT assay was used to detect antigen-specific T cells in fresh PBMCs (see Tan et al., 1999).

### Example 7

### Effects of MHC Class I and II Antigen Presentation Inhibitors on CDS8⁺ T cell Recognition.

The effects of various inhibitors, including proteasome inhibitors, MHC class II antigen processing inhibitor, protein synthesis inhibitors, and protease inhibitors on CD8⁺ T cell activity were examined in HEK293, 1359mel and LCL 111 target cells expressing HLA-B8 plus EBNA1-GFP or EBNA1 genes. Target cells were incubated in the absence or presence of various concentrations of inhibitors for different period of time, depending on the type of inhibitors used. The cells were then washed and counted and co-cultured with T cells overnight for IFN-γ release assays. The solvents used to dissolve the inhibitors, such as DMSO, methanol and ethanol were also used as controls. Melanoma derived TIL 102 CD4⁺ T cell recognition of 102mel tumor cells was used to demonstrate the specificity of lactacystin and chloroquine inhibitors of MHC class I and II antigen presentation, respectively.

### Example 8

### Generation of Human T Cells Specific for EBNA1

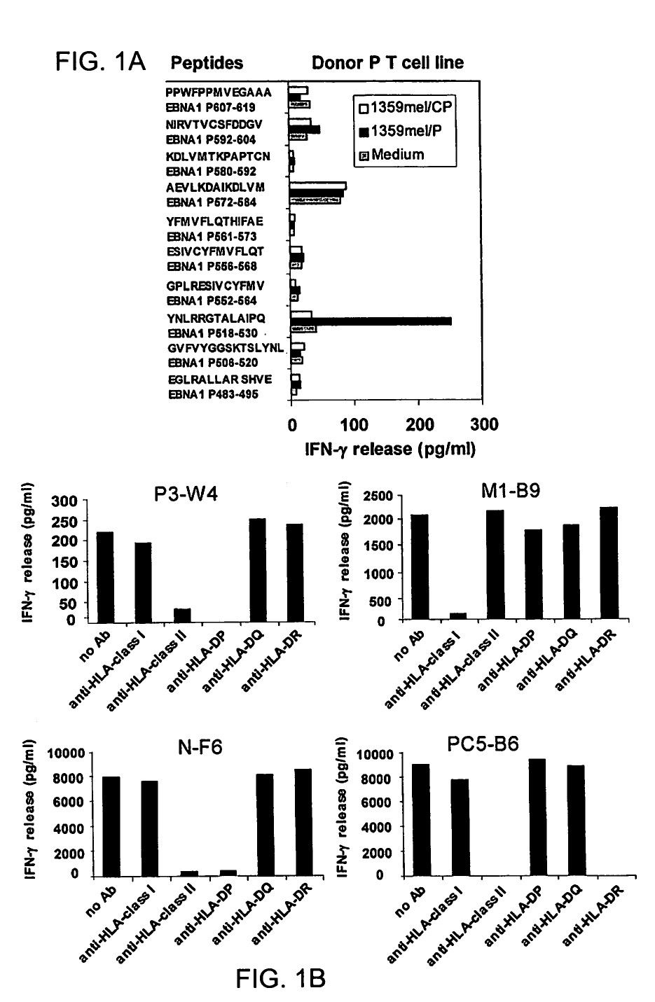

T-cell peptide presentation was identified by HLA-DR4 molecule, ten 13-15mer EBNA1 peptides were made, each predicted to have an HLA-DRB1*0401 binding motif by a computer-assisted algorithm, and the peptides were used to stimulate human PBMCs in vitro according to methods known in the art. See for example Zeng et al. 2000. Peptides other than those used for repeated stimulations were used as negative controls. For T cell recognition assays, peptides were pulsed onto HLA-DR4-matched 1359mel target cells at 15 μm concentration for 4 hours, washed twice, and co-cultured with T cells overnight. IFNγ release was measured in pg/ml. After three cycles of stimulation, T cells generated from PBMCs of each donor were tested for their ability to recognize HLA-DRB1*0401-matched 1359mel target cells pulsed with 10 individual peptides. A single 13mer peptide corresponding to EBNA1 P₅₁₈₋₅₃₀ (YNLRRGTALAIPQ) SEQ ID NO:1 elicited substantial secretion of IFN-γ from the T cell line P3-W4, which was obtained from the PBMCs of donor P (FIG. 1A). No peptide-specific T cell recognition was detected among the other T cell lines, nor among target cells pulsed with control peptides. T cell recognition assays were performed at an effector: target ratio of 1:1. All antibodies were used at a final concentration of 20 μg/ml each. Results are reported as the means of IFN-γ release in pg/ml from duplicate experiments. T cell lines that recognize MHC class I, HLA-DP or HLA-DR11 restricted antigens, respectively, were used for specificity and toxicity controls for mAb. M1-B9 CD8⁺ T cells recognize HLA-B8-restricted EBNA1peptide, N-F6 CD4⁺ T cells recognize a tumor antigen presented by HLA-DP molecule, and PC5-B6 CD4⁺ T cells respond to a tumor antigen presented by HLA-DR11 molecule.

T cell recognition was determined in the presence of antibodies against HLA class I, or class II, HLA-DP, -DQ and -DR molecules, which verified that T cell recognition of the EBNA1 P₅₁₈₋₅₃₀ SEQ ID NO:1 peptide was restricted by a HLA-DP molecule. These mAb were purified from culture supernatants of hybridoma cells and previously used for blocking T cell recognition of various T cell lines/clones. T cell recognition of the EBNA1 P₅₁₈₋₅₃₀ peptide by T cell line P3-W4 was specifically blocked by mAb against HLA class II and HLA-DP molecules, but not mAb by anti-HLA-DR, anti-HLA-DQ or anti-HLA class I molecules (FIG. 1B). Furthermore, anti-HLA-class II and HLA-DP mAb did not inhibit recognition of the EBNA1 peptide by CD8⁺M1-B9 T cells. Anti-HLA class II mAb blocked recognition of target cells by CD4⁺ N-F6 and PC5-B6 T cell clones, while anti-HLA-DP or anti-HLA-DR mAb could inhibit T cell recognition of antigens presented by the corresponding HLA-DP or HLA-DR molecules (FIG. 1B). These results indicate that human T cell line P3-W4 recognizes a peptide derived from EBNA1 presented by HLA-DP molecules.

### Example 9

### Characterization of T Cell Clones and Their Antigenic Peptides

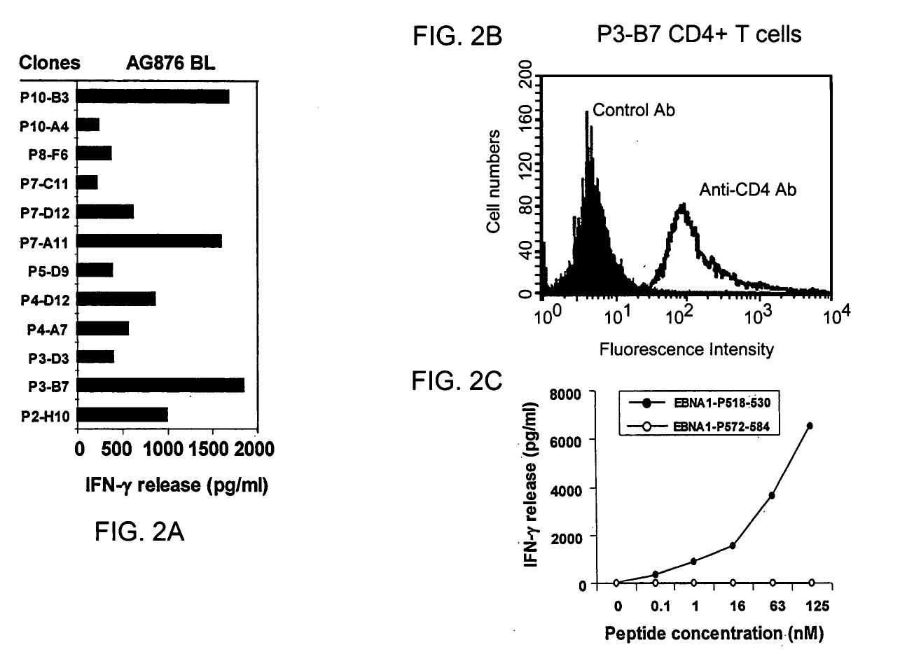

The P3-W4 T cell line was characterized and CD4⁺ T cell clones were generated by the limiting dilution method. Twelve CD4⁺ T cell clones specific for EBNA1-P₅₁₈₋₅₃₀ were successfully cloned and expanded. T cells were stained with anti-CD4-PE or anti-CD8-FITC, and after two washes, were analyzed by FACS. Positive staining for CD4 T cells is indicated by open symbols and control antibody staining is denoted by shaded symbols. C, EBNA1 P₅₁₈₋₅₃₀ peptide titration experiment for P3-B7 T cell recognition. Peptides at various concentrations were pulsed on autologous PBMCs for 3 h, then washed four times and used as target cells to stimulate T cells. A control peptide, EBNA1 P₅₇₂₋₅₈₄, was also used at various concentrations. IFN-γ secretion was measured in pg/ml. Although T cell clones were initially identified based on T cell activity of the peptide presented by 1359mel cells, 100-fold higher T cell activity was observed when autologous PBMCs were pulsed with the EBNA1-P₅₁₈₋₅₃₀ peptide compared to peptide-pulsed 1359mel cells, indicating that the antigen presenting molecules expressed on 1359mel cells are not the correct restriction molecules. T cell recognition of BL cell line (AG876) by different T cell clones was demonstrated (FIG. 2A). One of the T cell clones, designated P3-B7, was chosen, and FACS analysis showed that the P3-B7 T cells were CD4⁺0 T cells (FIG. 2B). Recognition of EBNA1 P₅₁₈₋₅₃₀ peptide by P3-B7 CD4⁺ T cells was blocked by antibody against HLA-DP molecules, indicating that the T cell clone closely resembles the original T cell line from which it was derived.

The minimum concentration of the EBNA1 P₅₁₈₋₅₃₀ peptide required for T cell recognition was determined, and the autologous donor P PBMCs were pulsed with the EBNA1 P₅₁₈₋₅₃₀ SEQ ID NO:1 peptide and used them as APCs. After four washes with serum-free RPMI medium to remove residual peptides, P3-B7 CD⁴⁺ T cells were co-cultured with peptide-pulsed target cells overnight. Culture supernatants were collected and T cell activity was determined on the basis of IFN-γ release from T cells. P3-B7 CD4⁺ T cells recognized the EBNA1 P₅₁₈₋₅₃₀ peptide at concentrations as low as 1 nM, and the T cell reactivity increased with increasing peptide concentrations (FIG. 2C). No T cell reactivity was observed against the control peptide, even at a concentration of 1 μM.

### Example 10

### Recognition of LCLs and EBV⁺ Burkitt's lymphoma cells by P3-B7 CD4⁺ T cells

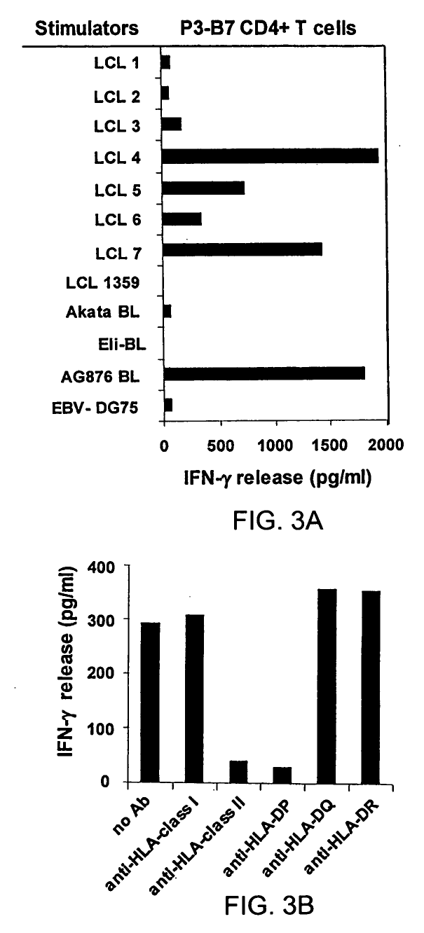

CD4⁺ T cells generated as described were capable of recognizing naturally processed peptides on LCLs and BL cells. Several LCLs and BL tumor cell lines were evaluated as target cells. T cell recognition was performed by co-culturing at an effector:target ratio of 1:1. IFN-γ secretion was measured by an ELISA kit. LCL7 and AG876 BL are DP3 positive based on DNA sequence analysis. HLA-DP typing of other cell lines is unknown. LCL1359 cells are HLA-DP3 negative. T cell recognition and antibody blocking assays were performed as described in Example 6. As shown in FIG. 3A, P3-B7 CD4⁺ T cells were capable of recognizing LCLs 4, 5, 6, 7 as well as AG876 BL tumor cells. Recognition of AG876 BL tumor cells by P3-B7 CD4⁺ T cells could be blocked by antibodies against MHC class II and HLA-DP molecules (FIG. 3B). Taken together, these results indicate that CD4⁺ T cells recognize a naturally processed peptide on the surface of EBV+BL cells in the context of HLA-DP molecules.

### Example 11

### Recognition of EBNA1 Protein by P3-B7 CD4⁺ T Cells in the Context of HLA-DP3

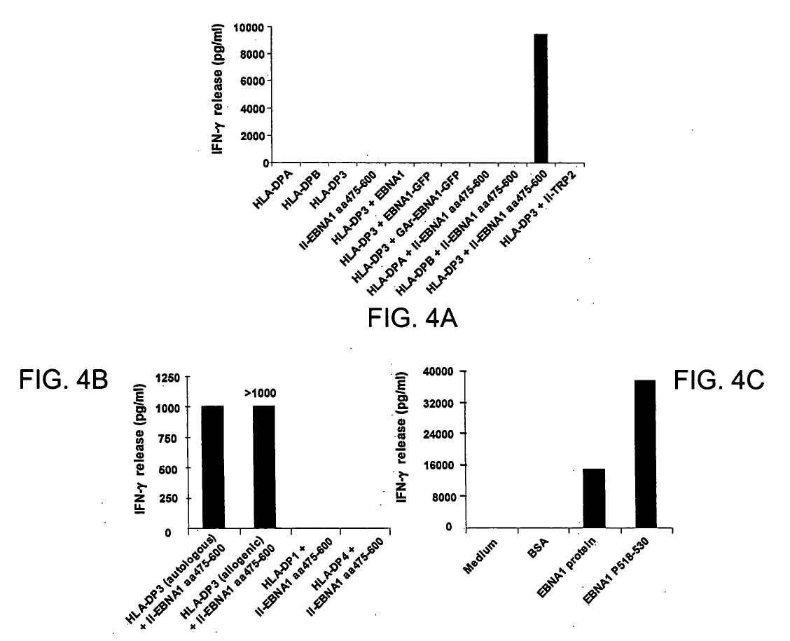

The restriction element for P3-B7 CD4⁺ T cells was determined, and the HLA-DP alleles were amplified from autologous PBMCs by RT-PCR, using HLA-DP-specific primers, and subcloned them into a pcDNA3.1/neo expression vector by methods known in the art. See for example Zeng et al. 2001. DNA sequence analysis revealed that HLA-DPA cDNA had 100% sequence homology to that published for HLA-DPA (DPA1*01031) and that HLA-DPB was almost identical to HLA-DPB1*0301 with a single nucleotide change from T to C at position 112 resulting in a substitution of histidine for tyrosine. DPA and DPB1*0301 cDNA were transfected into HEK293 cells together with Ii-EBNA1 or full-length EBNA1 cDNA. A protein concentration of 10 μg/ml of pure protein and bovine serum albumin control protein were loaded onto autologous PBMCs overnight. Protein-pulsed PBMCs were then washed four times with serum free RPMI medium and co-cultured with T cells overnight for IFN-γ release assay. The positive control EBNA1 P518-530 peptide at 15 μM was pulsed onto PBMCs from donor P for 3 h before co-culture with T cells. Background IFNγ release from the PBMCs was subtracted from the readout. As shown in FIG. 4A, T cells responded to HEK293 cells expressing HLA-DPA, HLA-DPB and Ii-EBNA1, demonstrating that HLA-DP3 (DPA1*01031/DPB1*0301) is the restriction element for the presentation of a peptide to T cells. T cells failed to recognize HEK293 cells transfected with HLA-DPA1*01031, HLA-DPB1*0301 and full-length EBNA1 cDNA, or HEK293 cells transfected with other cDNAs. This result indicates that without targeting of EBNA1 to the MHC class II pathway, HEK293 cells expressing HLA-DP3 molecules alone are not sufficient to process and present the EBNA1 peptide to T cells. To exclude the possibility that other HLA-DP alleles can present the EBNA peptide to T cells, HLA-DP 1, HLA-DP3 and HLA-DP4 cDNAs were co-transfected into 293 cells with Ii-EBNA, respectively. FIG. 4B shows that CD4⁺ T cells could recognize 293 cells transfected with HLA-DP3 and Ii-EBNA cDNAs, but did not respond to Ii-EBNA1 expressing 293 cells transfected with either HLA-DP1 or HLA-DP4 cDNAs. These results clearly demonstrate that HLA-DP3 is the antigen presenting molecule for CD4⁺ P3-B7 T cells.

EBNA1-specific CD4⁺ T cells are capable of recognizing the full-length EBNA1 protein. Autologous PBMCs were pulsed with the purified EBNA1 protein overnight, and CD4⁺ T cells were then co-cultured with protein-pulsed target cells for 18 h. An irrelevant protein, bovine serum albumin, was used as a control. As shown in FIG. 4C, P3-B7 CD4⁺ T cells specifically recognized autologous PBMCs pulsed with the full-length EBNA1 protein, but not bovine serum albumin.

### Example 12

### EBNA1 Protein of EBV is Processed and Presented to CD4⁺ T Cells in the Context of HLA-DP3 Molecule

EBNA1-specific CD4⁺ T cells recognize both the EBNA1 P₅₁₈₋₅₃₀ peptide and full-length EBNA1 protein pulsed on autologous PBMCs. Importantly, these T cells can recognize several HLA-DP3-matched LCLs and AG876 EBV⁺ BL tumor cells, indicating that the EBNA1 P₅₁₈₋₅₃₀ peptide is endogenously processed and then presented by DP3 molecules to T cells.

### Example 13

### Generation of MHC Class I-restricted T Cells Specific for EBNA1

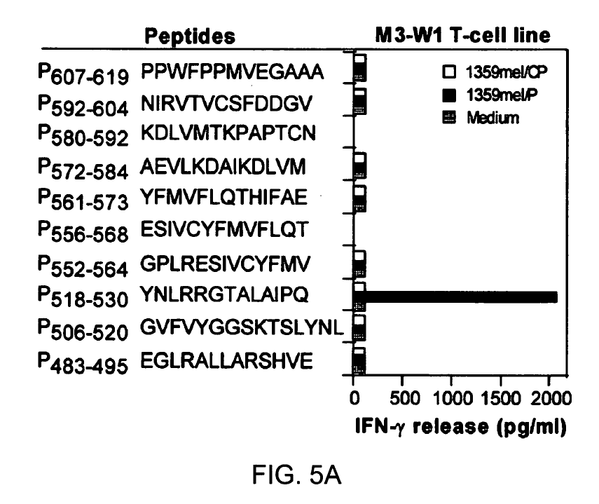

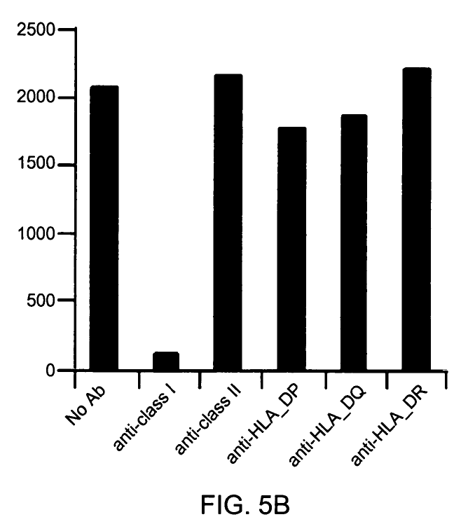

Several T cell lines were generated that were capable of stimulating T cells after co-culturing with EBNA1 P₅₁₈₋₅₃₀ (YNLRRGTALAIPQ) SEQ ID NO:1 peptide-pulsed 1359mel cells. Representative data from one of these cell lines, designated M3-W1, is shown in FIG. 5A. To determine the restriction element for T cell recognition, M3-W1 T cell activity was tested in response to the peptide-pulsed target cells in the presence of anti-MHC class I (HLA-A, B and C), anti-HLA-DR, anti-HLA-DP, and anti-HLA-DQ or isotype control antibodies. T cell recognition of the EBNA1 P₅₁₈₋₅₃₀ SEQ ID NO:1 peptide by T cell line M3-W1 was specifically blocked by an anti-MHC class I monoclonal antibody, but not by anti-HLA-DP, anti-DQ, anti-DR or isotype control antibodies (FIG. 5B). These results indicate that human M3-W1 T cells recognize a peptide derived from EBNA1 presented by HLA-class I molecules.

### Example 14

### Characterization of T Cell Clones and Their Antigenic Peptides

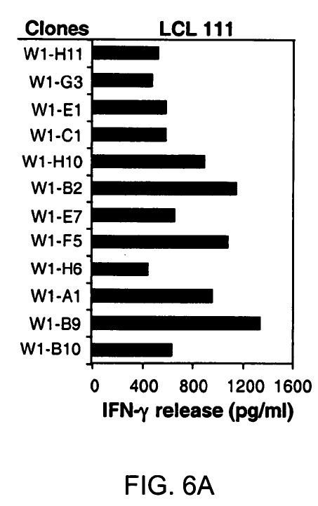

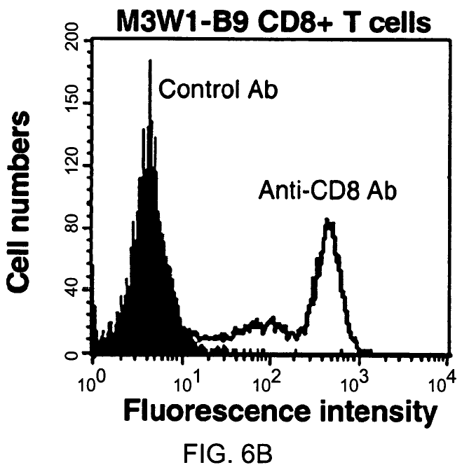

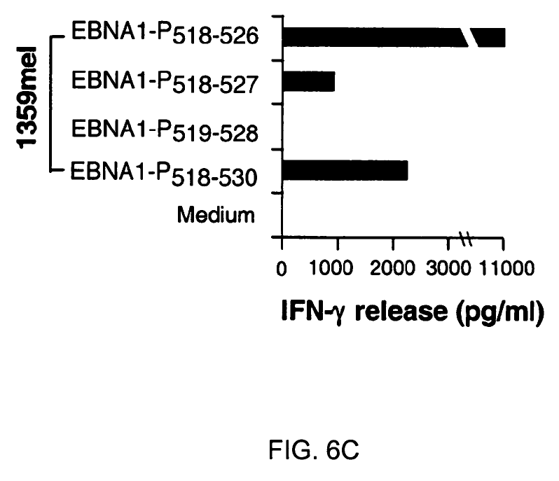

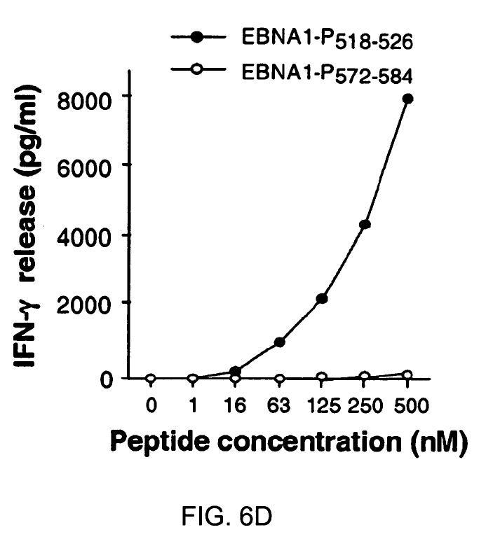

18 T cell clones were generated by limiting dilution methods. Recognition of LCL 111 by different T cell clones is presented in FIG. 6A. FACS analysis revealed that the M3W1-B9 T cells were CD8⁺ (FIG. 6B). Recognition of the EBNA1 P₅₁₈₋₅₃₀ SEQ ID NO:1 peptide by M3W1-B9 CD8⁺ T cells was also blocked by antibody against MHC-class I, but not by MHC class II or control antibodies, showing that these T cell clones resemble the bulk CD8⁺ T cell line.

Because the optimal peptide lengths for MHC class I molecules are generally 9-10 amino acids, three additional peptides: one 9-mer EBNA1-P₅₁₈₋₅₂₆ (YNLRRGTAL) SEQ ID NO:12 containing the HLA-B8 peptide binding motif, and two 10-mer peptides (EBNA1-P₅₁₈₋₅₂₇ SEQ ID NO:13 and EBNA1-P₅₁₉₋₅₂₈ SEQ ID NO:14) were generated from the parental EBNA1 P₅₁₈₋₅₃₀ peptide. These peptides were tested for their ability to stimulate M3W1-B9 CD8⁺ T cells. As shown in FIG. 6C, the EBNA1-P₅₁₈₋₅₂₆ peptide was recognized more readily by the M3W1-B9 CD8⁺ T cells than was the parental 13-mer peptide. By contrast, both EBNA1-P₅₁₈₋₅₂₇ (YNLRRGTALA) SEQ ID NO:13 and EBNA1-P₅₁₉₋₅₂₈ (NLRRGTALAI) SEQ ID NO:14 peptides exhibited lower or no activity for T cell recognition. Peptide titration experiments showed that T cell reactivity of the 9-mer EBNA1-P₅₁₈₋₅₂₆ peptide could be detected at a concentration of 63 nM (FIG. 6D). Thus, the 9-mer EBNA1-P₅₁₈₋₅₂₆ SEQ ID NO:12 peptide is recognized by the M3W1-B9 CD8⁺ T cells.

### Example 15

### EBNA1 Peptides are Naturally Processed and Presented to M3-W1-B9 CD8⁺ T Cells

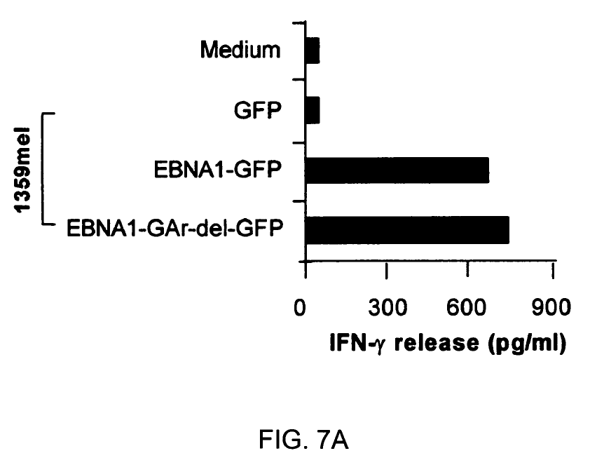

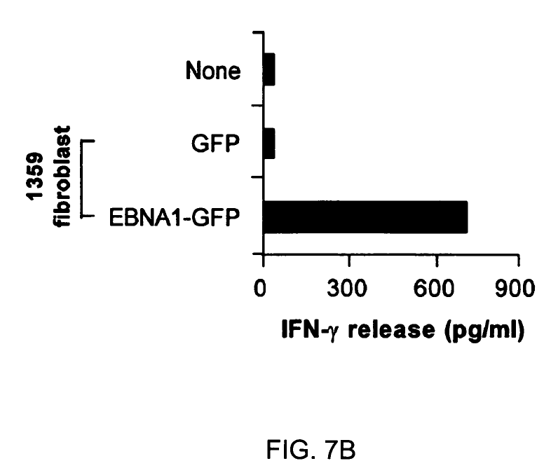

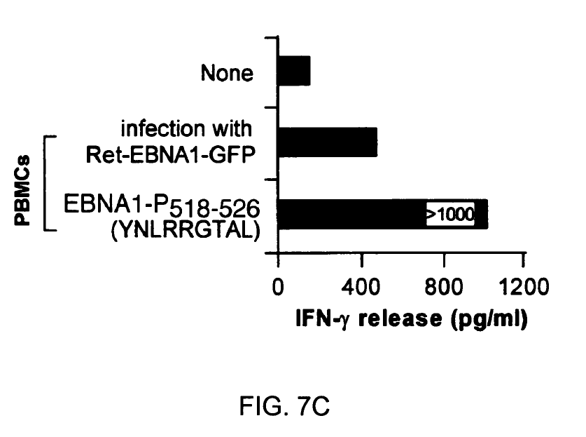

T cells were transfected plasmid DNAs carrying full-length EBNA1-GFP or EBNA1-GAr-del-GFP into 1359mel and 1359 fibroblast cells as targets for T cell recognition. As shown in FIG. 7A, M3W1-B9 CD8⁺ T cells recognized 1359mel/EBNA1-GFP and 1359mel/EBNA1-GAr-del-GFP target cells equally well, while no T cell reactivity was detected with 1359mel/GFP cells. The expression of EBNA1 as a fusion protein with GFP allowed us to monitor gene expression and transfection efficiency throughout the course of the experiments. CD8⁺ T cells also recognized 1359 fibroblast cells transfected with full-length EBNA1-GFP cDNA (FIG. 7B). The transfection efficiency of 1359mel and fibroblasts is about 10-15%. T cell recognition of 1359mel/EBNA1-GFP was also blocked by antibody against MHC class I, confirming that the recognition is MHC class I restricted. A retrovirus encoding EBNA1-GFP for introducing genes into PBMCs was constructed. T cell recognition of PBMCs infected with recombinant retrovirus increased 3-fold in terms of IFN-γ release from T cells compared with that of uninfected PBMCs (FIG. 7C). These results indicate that the MHC class I-restricted EBNA1 peptides are endogenously processed and presented to T cells.

### Example 16

### M3W1-B9 CD8⁺ T Cells Recognize EBNA1 Peptides Presented by HLA-B8 Molecules

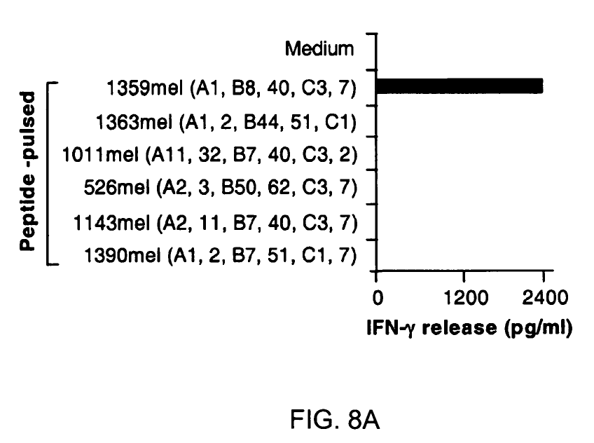

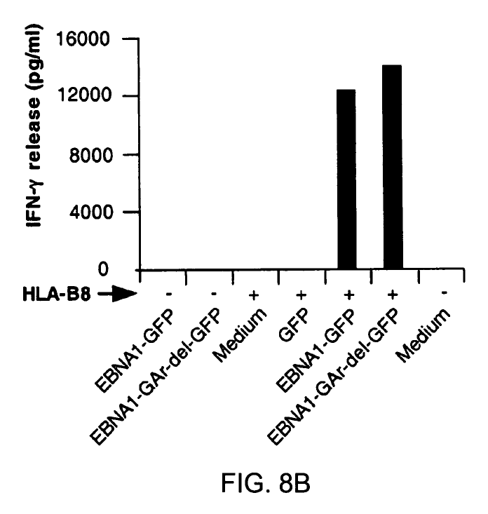

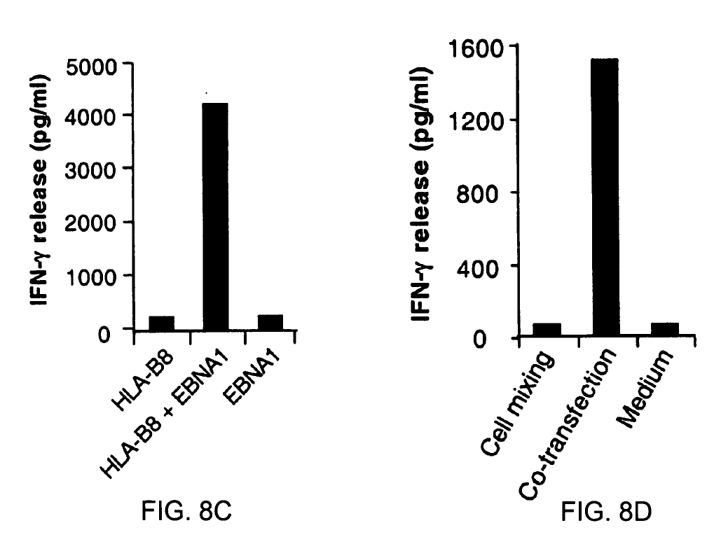

The 9-mer EBNA1 P₅₁₈₋₅₂₆ peptide was pulsed onto various MHC class I-positive melanoma cell lines and tested for T cell recognition. CD8⁺ T cells recognized 1359mel cells pulsed with the EBNA1-P₅₁₈₋₅₂₆ peptide, but not other cells pulsed with the same peptide (FIG. 8A), indicating that HLA-B8 is a putative restriction element for T cell recognition. HLA-B8 was cloned and the cDNA was transfected into HEK293 cells along with EBNA1-GFP or EBNA1-GAr-del-GFP and assessed their ability to present the antigenic peptide to T cells. While EBNA1-GFP, EBNA1-GAr-del-GFP or HLA-B8 expressed alone in HEK293 cell did not stimulate T cell responses, HEK293 cells transfected with EBNA1-GFP plus HLA-B8 or EBNA1-GAr-del-GFP plus HLA-B8 cDNAs strongly stimulated IFN-γ release from CD8⁺ T cells (FIG. 8B). Although T cell recognition of HEK293 cells transfected with EBNA1-GAr-del-GFP plus HLA-B8 cDNAs was slightly higher than that of HEK293 cells transfected with EBNA1-GFP and HLA-B8 cDNAs, there was no significant inhibitory effect of the Gly-Ala repeat domain on T cell responses (FIG. 8B). HLA-B8 is an antigen-presenting molecule for M3W1-B9 CD8⁺ T cells.

As shown in FIG. 8C, HEK293 cells transfected with EBNA1 plus HLA-B8 cDNA strongly stimulated IFN-γ release from CD8⁺ T cells, while HEK293 cells transfected with either one alone failed to stimulate T cell response. The native form of EBNA1 protein, like the GFP-tagged EBNA1, can be processed and presented to CD8⁺ T cells by the HLA-B8 molecules.

It is well known that dendritic cells have the capacity to capture and deliver exogenous antigens into the MHC class I processing pathway (Steinman et al., 2001 and Heath et al., 2001.). HEK293 cells were transfected with HLA-B8 cDNA or EBNA1-GFP cDNA separately, and then mixed (1:1) together as target cells in a T cell assay. As shown in FIG. 8D, M3W1-B9 CD8⁺ T cells did not respond to the mixed cells of transfected HEK293/HLA-B8 and HEK293/EBNA1-GFP, but they actively recognized HEK293 cells co-transfected with HLA-B8 and EBNA1-GFP cDNAs. Co-expression of HLA-B8 and EBNA1 in the same HEK293 cells is required for T cell recognition by M3W1-B9 CD8⁺ T cells, and that HEK293 cells are not capable of cross-presenting EBNA1 antigen to T cells.

### Example 17

### Recognition and Lysis of EBV⁺ LCL Cells by M3W1-B9 T Cells

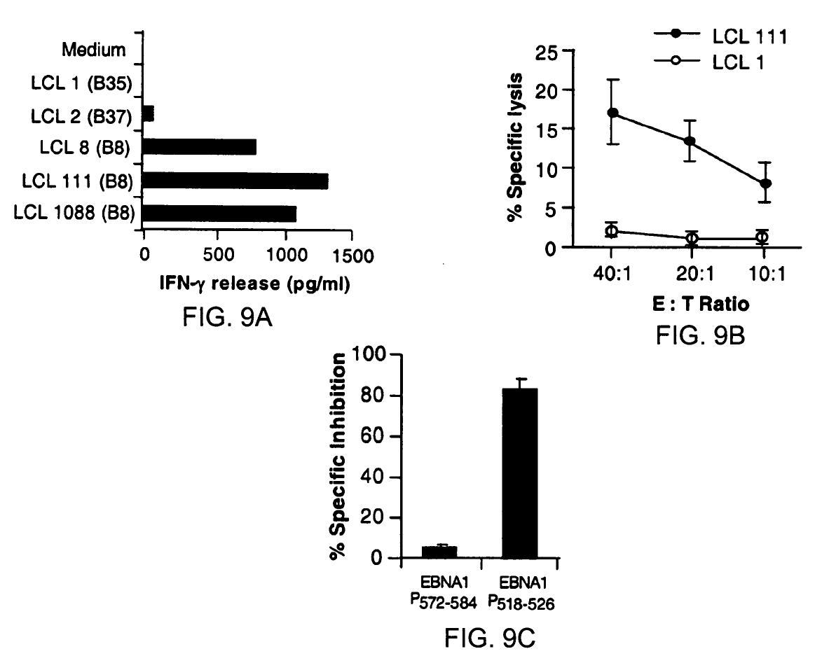

Chromium release assays were undertaken in which CD8⁺ T cells specifically lysed LCL 111 cells, but not the HLA mismatched LCL1 cells (FIG. 9B). Cold target inhibition experiments showed that the specific killing of chromium-labeled (hot) LCL111 target cells by the M3W1-B9 CD8⁺ T cells could be inhibited by the 9-mer EBNA1-P₅₁₈₋₅₂₆ peptide-pulsed unlabeled (cold) LCL111 cells, but not by a control peptide-pulsed cold LCL111 cells (FIG. 9C). Taken together, these results indicate that the 9-mer EBNA1-P₅₁₈₋₅₂₆ peptide can specifically block the recognition and lysis of EBV-positive LCL111 cells by the M3W1-B9 CD8⁺ T cells, implying that a similar or identical EBNA1 peptide is endogenously processed and presented to T cells by HLA-B8 molecules on the surface of EBV-positive LCL cells by a mechanism that overrides the inhibitory effect of the Gly-Ala repeat domain on processing and presentation of EBNA1.

### Example 18

### Recognition of EBNA1-P₅₁₈₋₅₃₀ Peptide by EBNA1-specific CD4⁺ and CD8⁺ T Cells

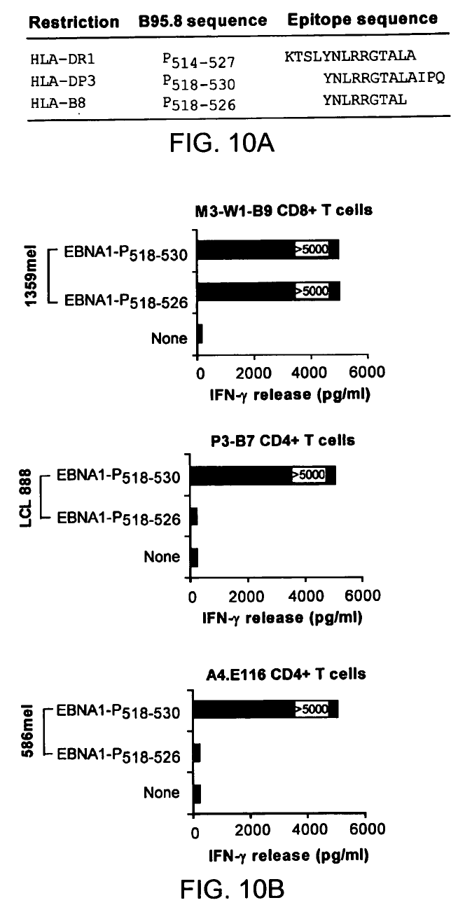

Since the HLA-B8-restricted EBNA1-P₅₁₈₋₅₂₆ peptide overlaps peptides presented by both HLA-DR1 and -DP3 molecules (FIG. 10A), it was determined if the same peptide could be recognized by EBNA1-specific CD4⁺ and CD8⁺ T cell clones. FIG. 10B shows that the EBNA1-P₅₁₈₋₅₃₀ peptide could be recognized by HLA-DR1- and -DP3-restricted CD4⁺ as well as M3W1-B9 CD8⁺ T cells when pulsed on their corresponding HLA-matched target cells. However, the 9-mer EBNA1-P₅₁₈₋₅₂₆ peptide was recognized only by CD8⁺ T cells, and not by HLA-DR1- and HLA-DP3-restricted CD4⁺ T cells, indicating that the short peptide is specific for CD8⁺ T cells. These results show that the 13mer EBNA1-P₅₁₈₋₅₃₀ peptide has a dual function, as it can stimulate both CD4⁺ and CD8⁺ T cell responses.

### Example 19

### Determination of EBNA1-specific HLA-IB8-restricted CD8⁺ T Cells in Other Donor PBMCs

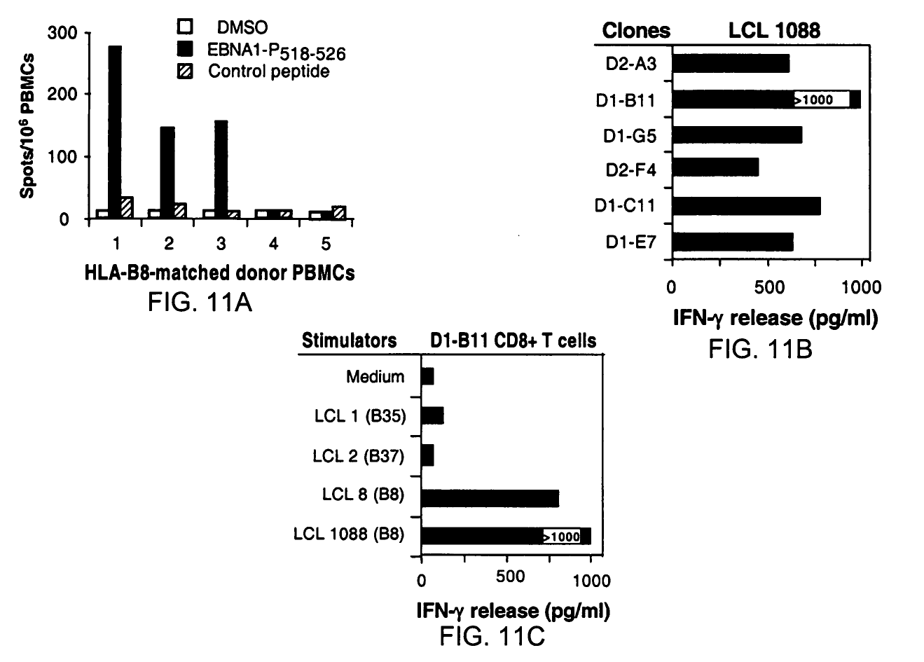

Five donor PBMCs were obtained for ELISPOT assays using EBNA1-P₅₁₈₋526 peptide-pulsed target cells. A NY-ESO-1 peptide-pulsed target cells served as a specific control. Three of the 5 donor PBMCs (donors # 1, 2 and 3) express HLA-B8 molecules and are serum positive for EBV, while donor #4 is positive for HLA-B8 expression, but is serum negative for EBV. A mismatched donor PBMC (donor # 5) served as a negative control. Three of the 4 HLA-B8-positive donor PBMCs specifically responded to the EBNA1-P₅₁₈₋₅₂₆ peptide (FIG. 11A). By contrast, neither PBMCs from HLA-B8 positive but seronegative nor HLA-B8-negative donors responded to the EBNA1-P₅₁₈₋₅₂₆ peptide (FIG. 11A). These results illustrate that EBNA1-specific, HLA-B8-restricted CD8⁺ T cells are commonly present in HLA-B8 positive EBV-infected individuals. To further test whether these EBNA1-specific, HLA-B8-restricted CD8⁺ T cells are capable of recognizing EBV positive LCL cells, T cell clones from the PBMCs of donor #3 were established by limiting dilution methods. Recognition of HLA-B8 expressing LCL 1088 by these T cell clones is shown in FIG. 11B. One T clone, D1-B11, was selected for further testing of its ability to recognize HLA-B8 positive EBV-positive LCL cells. These T cells exhibited strong T cell reactivity against HLA-B8-positive LCLs 8 and 1088, but did not respond to HLA-B8 negative LCL 1 and 2 cells (FIG. 11C). PBMCs of EBV infected donors expressing HLA-B8 molecules contain EBNA1-specific CD8⁺ T cells that are capable of recognizing HLA-B8 positive LCL targets.

### Example2

### Inhibition of T Cell Recognition by Proteasome Inhibitors

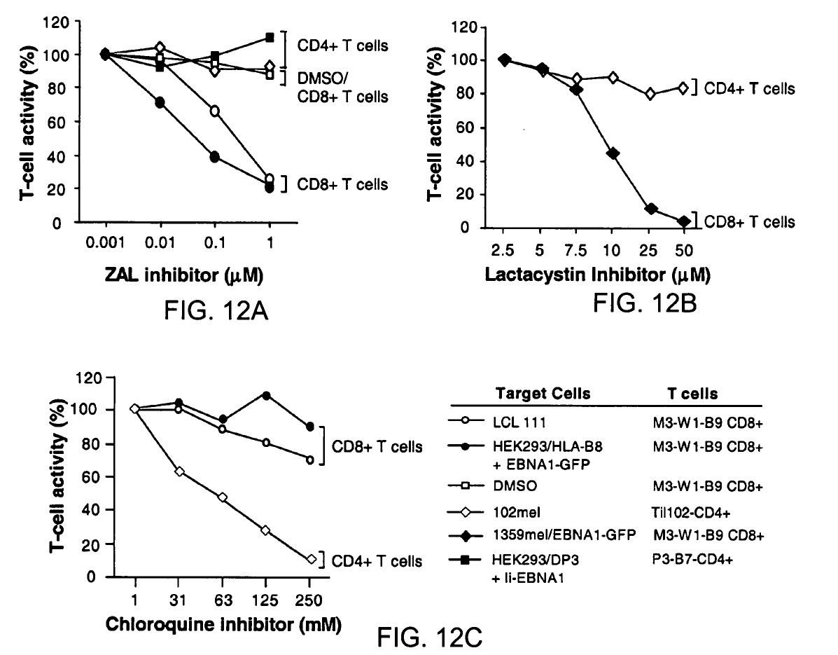

HEK293 cells were transfected with EBNA1-GFP and HLA-B8 cDNAs, and then treated them with different concentrations of ZAL and lactacystin, specific inhibitors of proteasomes. When antigen-specific CD8⁺ T cells were then co-cultured with target cells to evaluate T cell responses, T cell reactivity of HEK293 transfected with HLA-B8 plus EBNA1-GFP cDNAs by the M3W1-B9 CD8⁺ T cells decreased with increasing concentrations of proteasome inhibitors ZAL, but not in the presence of control DMSO (FIG. 12A). Similarly, T cell reactivity against LCL111 cells was inhibited with increasing concentration of ZAL inhibitor. To exclude the potential nonspecific effect of ZAL on T cell recognition, the effects of ZAL inhibitor were tested on melanoma-reactive TIL102-CD4⁺ T cells, which recognize a MHC class II-restricted epitope on the cell surface of 102mel tumor cells, and of P3-B7 CD4⁺ T cells, which recognize HLA-DP3 expressing HEK293 cells transfected with Ii-EBNA1 as target cells. The ZAL inhibitor did not have any inhibitory effect on recognition of target cells by TIL102 CD4⁺ T cells or P3-B7 T CD4⁺ cells (FIG. 12A). The effects of lactacystin, another specific proteasome inhibitor, were also tested on T cell recognition. As shown in FIG. 8B, recognition of 1359mel/EBNA1-GFP cells by M3-W1-B9 CD8⁺ T cell was decreased with increasing concentrations of lactacystin, while no inhibitory effect was observed with TIL102 CD4⁺ T cell recognition of 102mel target cells. These results indicate that the inhibitory effect of ZAL and lactacystin is specific for the presentation of HLA-B8-restricted epitope to CD8⁺ T cells, but not for MHC class II antigen processing and presentation.

T cell recognition of HEK293 cells transfected with HLA-B8 plus EBNA1-GFP cDNAs or LCL111 by M3W1-B9 CD8⁺ T cells was not significantly affected after the target cells were treated with different concentrations of chloroquine (FIG. 12C). By contrast, T cell activity of TIL102-CD4⁺ T cells against 102mel cells was significantly inhibited by chloroquine, indicating that chloroquine can specifically inhibit MHC class II, but not class I pathway for antigen presentation. Processing and presentation of the HLA-B8-restricted EBNA1 peptides require the participation of proteasomes in the MHC class I pathway.

### Example 21

### ELA-B8-Restricted EBNA1 Epitope is Derived From Defective Ribosomal Products

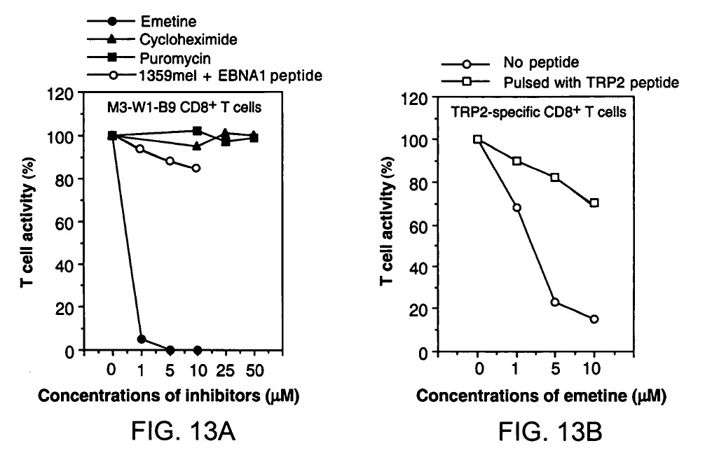

HLA-B8 expressing HEK293/EBNA1-GFP cells were treated with different concentrations of irreversible (emetine) and reversible (puromycin and cycloheximide) protein synthesis inhibitors for 1 h. After washing to remove inhibitors, the treated cells were co-cultured with antigen-specific CD8⁺ T cells. FIG. 13A shows that recognition of target cells by M3W1-B9 CD8⁺ T cells was significantly (94%) inhibited at a low concentration (1 μM), and completely inhibited at a 5 μM concentration of the irreversible inhibitor emetine. By contrast, no inhibitory effect was observed when HLA-B8 expressing 293/EBNA1-GFP cells were treated with reversible inhibitors (puromycin and cycloheximide), respectively. Since blocking protein synthesis should rapidly decrease the peptide supply required for the export of MHC class I molecules from the ER to the cell surface as well as synthesis of MHC class I molecules, the treatment of cells with emetine would decrease the overall antigen presentation by MHC class I molecules.

HLA-B8 positive 1359mel cells were pulsed with the EBNA1-P₅₁₈₋₅₂₆ peptide after the cells were treated with different concentrations of emetine and washed. T cell recognition of peptide-pulsed target cells was slightly inhibited (FIG. 13A), indicating that effect of emetine on MHC class I molecules could not account for the inhibition of T cell recognition. 1363mel cells (TRP2⁺ and HLA-A2⁺) were treated with different concentrations of emetine, and then tested for their ability to stimulate TRP2-specific CD8⁺ T cells. As shown in FIG. 9B, the inhibition of recognition of 1363mel target cells by TRP2-specific CD8⁺ T cells increased with the increasing concentrations of emetine. However, there was only 30% inhibition of T cell recognition at 1 μM emetine treatment compared with over 90% inhibition of EBNA1-specific CD8⁺ T cells at the same concentration of emetine (FIG. 13A). As expected, T cell recognition of the TRP2 peptide-pulsed target cells after the emetine treatment was slightly inhibited (FIG. 13B). Treatment of target cells with emetine at an 1 μM concentration resulted in 20% inhibition of EBNA1-specific P3-B7 CD4⁺ T cell recognition. Taken together, these results indicate that new protein synthesis is necessary and required for the generation of the HLA-B8-restricted epitope for T cell recognition, thus indicating that DRiPs are the primary source of CD8⁺ T cell peptides. On the other hand, the processing and presentation of the TRP2 and CD4⁺ T cell EBNA1 epitopes are less dependent on the production of short-lived DRiPs.

### Example 22

### Serine Proteases are Involved in the Generation of T Cell Epitopes

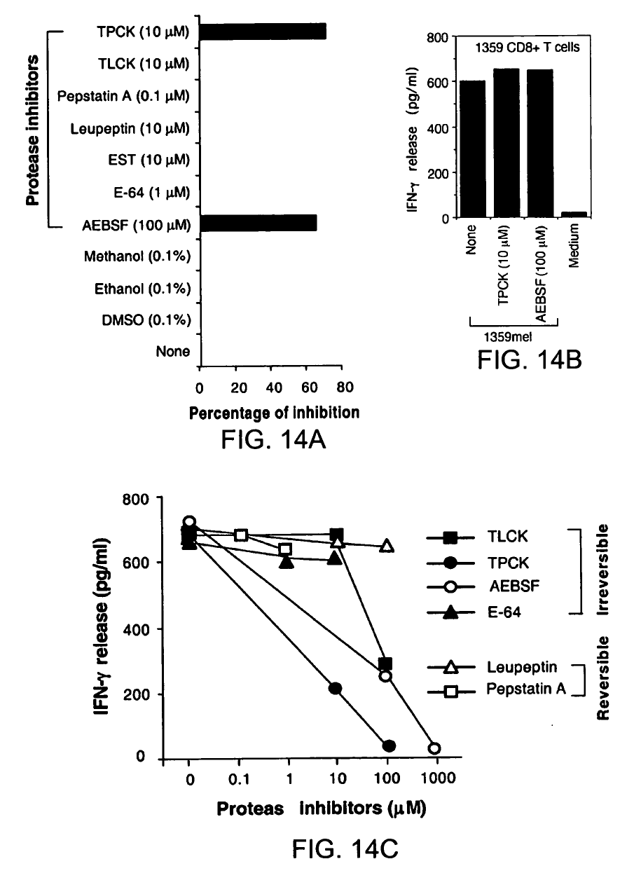

Seven protease inhibitors were selected for further testing. FIG. 14A shows that two such protease inhibitors, TPCK and AEBSF, significantly blocked CD8⁺ T cell recognition of the HLA-B8-expressing HEK293/EBNA1-GFP target cells when treated for 2 h. None of other protease inhibitors were effective, even after treatment for 2 or 8 h. To test whether TPCK or AEBSF affected T cell recognition of other antigens, 1359mel were treated with the same concentrations of TPCK or AEBSF as described in FIG. 14A. No inhibition was observed for recognition of 1359mel cells by 1359mel-specific CD8⁺ T cells (FIG. 14B), illustrating that both inhibitors are specific for processing and presentation of the HLA-B8-restricted EBNA1 epitope. TPCK or AEBSF inhibited T cell recognition of target cells in a dose-dependent manner (FIG. 14C), while TLCK produced a partial inhibitory effect when used at a high concentration. Although TLCK and TPCK have similar specificities, TLCK is unstable in solution according to the manufacturer. AEBSF is an irreversible, specific inhibitor of serine proteases, while TPCK is an irreversible inhibitor of chymotrypsin and many other serine and cysteine proteases. These results indicate that serine proteases are also required for the processing of the HLA-B8-restricted T cell epitope.

### Example 23

### Conclusions

The CD4⁺ T cells generated as described herein recognized peptides presented by HLA-DP3 molecules. The EBNA1 peptide-specific HLA-DP3-restricted CD4⁺ T cells recognize BL tumor cells. By contrast, the previously reported EBNA1 peptide-specific, HLA-DR1-DR11 or DR15-restricted CD4⁺ T cells failed to recognize EBV-positive autologous LCLs or BL tumor cells, limiting their potential therapeutic value. While the HLA-DP3-restricted EBNA1 P₅₁₈₋₅₃₀ peptide presented here overlaps with the DR1-restricted EBNA1 P₅₄₁₋₅₂₇ peptide, this is a novel HLA-DP-restricted peptide. Thus, the data indicate that CD4⁺ T cells recognize EBV-positive LCLs and BL tumor cells.

Additionally, the results presented here demonstrate that CD8⁺ T cells generated from in vitro peptide stimulation recognize 13-mer and 9-mer EBNA1 peptides, and that the 9-mer EBNA1 peptide is the more effectively recognized of the two (FIG. 6C). The data showed that EBNA1-specific CD8⁺ T cells can recognize EBNA1/HLA-B8 expressed by HEK293 and 1359mel, and LCL111 cells (FIGS. 7 to 9), indicating that EBNA1 can be endogenously processed and presented by HLA-B8 molecules for T cell recognition. HLA-B8 and EBNA1 must be expressed in the same HEK293 cells in order for EBNA1 to be processed and presented by HLA-B8 molecule.

The EBNA1 P₅₁₈₋₅₂₆ peptide-specific CD8+ T-cells are readily detectable in the HLA-B8 expressing donor PBMCs. HLA-B8-restricted CD8⁺ T cells established from these HLA-B8 ⁺ donors responded functionally to the peptide-pulsed target cells as well as HLA-B8 matched LCLs.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.

U.S. Patents and Patent Publications 5,869,453 20030175300 20030175300 20020150590

Non-patent Publications

Blake, N., S. Lee, I. Redchenko, W. Thomas, N. Steven, A. Leese, P. Steigerwald-Mullen, M.G. Kurilla, L. Frappier, and A. Rickinson. 1997. Human CD8+ T cell responses to EBV EBNA1: HLA class I presentation of the (Gly-Ala)-containing protein requires exogenous processing. Immunity 7:791-802.

Blake, N., T. Haigh, G. Shaka'a, D. Croom-Carter, and A. Rickinson. 2000. The importance of exogenous antigen in priming the human CD8+ T cell response: lessons from the EBV nuclear antigen EBNA1. J. Immunol. 165:7078-7087.

Chaux, P., Vantomme, V., Stroobant, V., Thielemans, K., Corthals, J., Luiten, R., Eggermont, A. M., Boon, T., and van der Bruggen, P. Identification of MAGE-3 Epitopes Presented by HLA-DR Molecules to CD4(+) T Lymphocytes. J Exp Med. 189: 767-778, 1999.

Frappier, L. and O'Donnell, M. Overproduction, purification, and characterization of EBNA1, the origin binding protein of Epstein-Barr virus. J Biol Chem. 266: 7819-7826., 1991.

Germain, R. N. 1994. MHC-dependent antigen processing and peptide presentation: providing ligands for T lymphocyte activation. Cell 76:287-299.

Gromme, M., F. G. C. M. Uytdehaag, H. Janssen, J. Calafat, R. S.v. Binnendijk, M. J. H. Kenter, A. Tulp, D. Verwoerd, and J. Neefjes. 1999. Recycling MHC class I molecules and endosomal peptide loading. Proc. Natl. Acad. Sci. USA 96:10326-10331.

Heath, W. R., and F. R. Carbone. 2001. Cross-presentation, dendritic cells, tolerance and immunity. Annu. Rev. Immunol. 19:47-64.

Heslop, H. E., Perez, M., Benaim, E., Rochester, R., Brenner, M. K., and Rooney, C. M. Transfer of EBV-specific CTL to prevent EBV lymphoma post bone marrow transplant. J Clin Apheresis. 14: 154-156, 1999.

Kalams, S. A. and Walker, B. D. The critical need for CD4 help in maintaining effective cytotoxic T lymphocyte responses. J Exp Med. 188: 2199-2204, 1998.

Khanna, R., D. J. Moss, and S. R. Burrows. 1999. Vaccine strategies against Epstein-Barr virus-associated diseases: lessons from studies on cytotoxic T-cell-mediated immune regulation. Immunol. Rev. 170:49-64.

Khanna, R., Burrows, S. R., Steigerwald-Mullen, P. M., Thomson, S. A., Kurilla, M. G., and Moss, D. J. Isolation of cytotoxic T lymphocytes from healthy seropositive individuals specific for peptide epitopes from Epstein-Barr virus nuclear antigen 1: implications for viral persistence and tumor surveillance. Virology. 214: 633-637, 1995.

Khanna, R., Moss, D. J., and Burrows, S. R. Vaccine strategies against Epstein-Barr virus-associated diseases: lessons from studies on cytotoxic T-cell-mediated immune regulation. Immunol Rev. 170: 49-64, 1999.

Kieff, E. Epstein-Barr virus-increasing evidence of a link to carcinoma. N Engl J Med. 333: 724-726, 1995.

Leen, A., Meij, P., Redchenko, I., Middeldorp, J., Bloemena, E., Rickinson, A., and Blake, N. Differential immunogenicity of Epstein-Barr virus latent-cycle proteins for human CD4(+) T-helper 1 responses. J Virol. 75: 8649-8659, 2001.

Levitskaya, J., A. Sharipo, A. Leonchiks, A. Ciechanover, and M. G. Masucci. 1997. Inhibition of ubiquitin/proteasome-dependent protein degradation by the Gly-Ala repeat domain of the Epstein-Barr virus nuclear antigen 1. Proc. Natl. Acad. Sci. USA 94:12616-12621.

Levitskaya, J., M. Coram, V. Levitsky, S. Imreh, P. M. Steigerwald-Mullen, G. Klein, M. G. Kurilla, and M. G. Masucci. 1995. Inhibition of antigen processing by the internal repeat region of the Epstein-Barr virus nuclear antigen-1. Nature 375:685-688.

Munz, C., Bickham, K. L., Subklewe, M., Tsang, M. L., Chahroudi, A., Kurilla, M. G., Zhang, D., O'Donnell, M., and Steinman, R. M. Human CD4(+) T lymphocytes consistently respond to the latent Epstein-Barr virus nuclear antigen EBNA1. J Exp Med. 191: 1649-1660, 2000.

Nikiforow, S., Bottomly, K., and Miller, G. CD4⁺ T-cell effectors inhibit Epstein-Barr virus-induced B-cell proliferation. J Virol. 75: 3740-3752, 2001.

Paludan, C., Bickham, K., Nikiforow, S., Tsang, M. L., Goodman, K., Hanekom, W. A., Fonteneau, J. F., Stevanovic, S., and Munz, C. Epstein-Barr Nuclear Antigen 1-Specific CD4(+) Th1 Cells Kill Burkitt's Lymphoma Cells. J Immunol. 169: 1593-1603, 2002

Reits, E. A., J. C. Vos, M. Gromme, and J. Neefjes. 2000. The major substrates for TAP in vivo are derived from newly synthesized proteins. Nature 404: 774-778.

Rickinson, A. B. and Moss, D. J. Human cytotoxic T lymphocyte responses to Epstein-Barr virus infection. Annu Rev Immunol. 15: 405-431, 1997.

Rock, K. L., I. A. York, T. Saric, and A. L. Goldberg. 2002. Protein degradation and the generation of MHC class I-presented peptides. Adv. Immunol. 80:1-70.

Roskrow, M. A., Suzuki, N., Gan, Y., Sixbey, J. W., Ng, C. Y., Kimbrough, S., Hudson, M., Brenner, M. K., Heslop, H. E., and Rooney, C. M. Epstein-Barr virus (EBV)-specific cytotoxic T lymphocytes for the treatment of patients with EBV-positive relapsed Hodgkin's disease. Blood. 91: 2925-2934, 1998.

Rowe, M., Rowe, D. T., Gregory, C. D., Young, L. S., Farrell, P. J., Rupani, H., and Rickinson, A. B. Differences in B cell growth phenotype reflect novel patterns of Epstein-Barr virus latent gene expression in Burkitt's lymphoma cells. Embo J. 6: 2743-51, 1987.

Schubert, U., L. C. Anton, J. Gibbs, C. C. Norbury, J. W. Yewdell, and J. R. Bennink. 2000. Rapid degradation of a large fraction of newly synthesized proteins by proteasomes. Nature 404: 770-774.

Steinman, R. M., and M. Dhodapkar. 2001. Active immunization against cancer with dendritic cells: the near future. Int. J Cancer 94:459-473.

Tan, L. C., N. Gudgeon, N. E. Annels, P. Hansasuta, C. A. O'Callaghan, S. Rowland-Jones, A. J. McMichael, A. B. Rickinson, and M. F. Callan. 1999. A re-evaluation of the frequency of CD8+ T cells specific for EBV in healthy virus carriers. J Immunol. 162:1827-1835.

Tellam, J., Sherritt, M., Thomson, S., Tellam, R., Moss, D. J., Burrows, S. R., Wiertz, E., and Khanna, R. Targeting of EBNA1 for rapid intracellular degradation overrides the inhibitory effects of the Gly-Ala repeat domain and restores CD8+ T cell recognition. J Biol Chem. 276: 33353-33360, 2001.

Voo, K. S., T. Fu, H. E. Heslop, M. K. Brenner, C. M. Rooney, and R.-F. Wang. 2002. Identification of HLA-DP3-restricted Peptides from EBNA1 Recognized by CD4+ T Cells. Cancer Res. 62:7195-7199.

Wang, R.-F., Johnston, S. L., Zeng, G., Schwartzentruber, D. J., and Rosenberg, S. A. A breast and melanoma-shared tumor antigen: T cell responses to antigenic peptides translated from different open reading frames. J. Immunol. 161: 3596-3606, 1998.

Wang, R. F. and Rosenberg, S. A. Human tumor antigens for cancer vaccine development. Immunol Rev. 170: 85-100, 1999.

Wang, R.-F., Wang, X., Atwood, A. C., Topalian, S. L., and Rosenberg, S. A. Cloning genes encoding MHC class II-restricted antigens: mutated CDC27 as a tumor antigen. Science. 284: 1351-1354, 1999.

Wang, R.-F. The role of MHC class II-restricted tumor antigens and CD4+ T cells in antitumor immunity. Trends in Immunology. 22: 269-276, 2001.

Zajac, A. J., Murali-Krishna, K., Blattman, J. N., and Ahmed, R. Therapeutic vaccination against chronic viral infection: the importance of cooperation between CD4⁺ and CD8⁺ T cells. Curr Opin Immunol. 10: 444-449, 1998.

Zarour, H. M., Maillere, B., Brusic, V., Coval, K., Williams, E., Pouvelle-Moratille, S., Castelli, F., Land, S., Bennouna, J., Logan, T., and Kirkwood, J. M. NY-ESO-1 119-143 is a promiscuous major histocompatibility complex class II T-helper epitope recognized by Th1- and Th2-type tumor- reactive CD4⁺ T cells. Cancer Res. 62: 213-218, 2002.

Zeng, G., Touloukian, C. E., Wang, X., Restifo, N. P., Rosenberg, S. A., and Wang, R.-F. Identification of CD4⁺ T cell epitopes from NY-ESO-1 presented by HLA-DR molecules. J. Immunol. 165:1153-1159,2000.

Zeng, G., Wang, X., Robbins, P. F., Rosenberg, S. A., and Wang, R-F. CD4⁺ T cell recognition of MHC class II-restricted epitopes from NY-ESO-1 presented by a prevalent HLA-DP4 allele: association with NY-ESO-1 antibody production. Proc. Natl. Acad. Sci. U.S.A. 98: 3964-3969, 2001.

What is claimed is:

1. An EBNA1 epitope comprising SEQ ID NO:1 or SEQ ID NO:12.

2. The epitope of claim 1, wherein the epitope is presented by MHC class I or MHC class II molecules.

3. The epitope of claim 2, wherein the epitope presented by MHC class II molecules is HLA-DP3-restricted.

4. The epitope of claim 2, wherein the epitope presented by MHC class I molecules is HLA-B8-restricted.

5. The epitope of claim 1, wherein the epitope is presented by an Epstein-Barr infected B lymphocyte.

6. The epitope of claim 1, wherein the epitope stimulates both CD4+ and CD8+ T cells.

7. A T cell comprising a CD4+ T cell, which is specific to the epitope of claim 1.

8. A T cell comprising a CD8+ T cell, which is specific to the epitope of claim 1.

9. A method for stimulating T cells specific for an EBNA1 epitope comprising the step of:
contacting said T cells under conditions and for a time sufficient to permit the stimulation of said T cells, with at least one component selected from the group consisting of: the EBNA1 epitope, an antigen-presenting cell that recombinantly expresses and presents the EBNA1 epitope, and an antigen-presenting cell that expresses the endogenously processed EBNA1 epitope.

10. The method of claim 9, wherein said antigen-presenting cell comprises a B lymphocyte.

11. The method of claim 9, wherein said antigen-presenting cell comprise a dendritic cell.

12. An isolated T cell population comprising T cells prepared according to the method of claim 9.

13. An immunological composition comprising the T cells prepared according to the method of claim 9.

14. A method for stimulating an immune response in a patient, comprising the step of:
administering to the patient an immunological composition comprising the T cell population prepared according to the method of claim 9.

15. A method for expanding T cells specific for an EBNA1 epitope comprising the step of:
contacting said T cells under conditions and for a time sufficient to permit the expansion of said T cells, with at least one component selected from the group consisting of: the EBNA1 epitope, an antigen-presenting cell that recombinantly expresses and presents the EBNA1 epitope, and an antigen-presenting cell that expresses the endogenously processed EBNA1 epitope.

16. A method of treating human lymphoproliferative disorders, wherein said immunotherapy comprises the step of:
administering to the patient a composition comprising T cells that have been contacted under conditions and for a time sufficient to permit the stimulation or expansion of said T cells, with at least one component selected from the group consisting of: an EBNA1 epitope, an antigen-presenting cell that recombinantly expresses and presents the EBNA1 epitope, and an antigen-presenting cell that expresses the endogenously processed EBNA1 epitope.

17. The method of claim 16, wherein the lymphoproliferative disorder is Burkitt's lymphoma.

18. The method of claim 16, wherein the lymphoproliferative disorder is Hodgkin's lymphoma.

19. A fusion protein comprising SEQ ID NO:1 or SEQ ID NO:12 and a domain that enhances MHC class II processing.

20. The protein of claim 19, wherein the domain that enhances MHC class II processing comprises the invariant chain protein.

21. A recombinant expression vector comprising an isolated nucleic acid sequence encoding SEQ ID NO:1 or SEQ ID NO:12 and at least one gene encoding a co-immunostimulatory molecule.

22. A method of treating a person infected with Epstein-Barr virus comprising the step of:
administering to said person a component selected from the group consisting of: a peptide comprising SEQ ID NO:1, a peptide comprising SEQ ID NO:12, T cells specific for SEQ ID NO:1, T cells specific for SEQ ID NO:12, and any combination thereof.

23. The method of claim 22, further comprising co-administration of at least one antigen-presenting cell.

24. A method for stimulating an immune response in a patient comprising the step of:
administering to said patient an immunological composition comprising at least one antigen presenting cell that presents an EBNA1 epitope comprising SEQ ID NO:1 or SEQ ID NO:12.

25. The method of claim 24, wherein the antigen presenting cell is a dendritic cell.

26. The method of claim 24, wherein the antigen presenting cell stimulates CD4+ or CD8+ T cells or any combination thereof.
